(19) United States
(12) Patent Application Publication
(10) Pub. No.: US 2018/0133463 A1
(43) Pub. Date: May 17, 2018

(54) ELECTRODE FOR SENSING, PACING, AND DEFIBRILLATION DEPLOYABLE IN THE MEDIASTINAL SPACE

(71) Applicant: CARDIAC PACEMAKERS, INC., St. Paul, MN (US)

(72) Inventor: G. Shantanu Reddy, Minneapolis, MN (US)

(73) Assignee: CARDIAC PACEMAKERS, INC., St. Paul, MN (US)

(21) Appl. No.: 15/815,051

(22) Filed: Nov. 16, 2017

Related U.S. Application Data
(60) Provisional application No. 62/423,630, filed on Nov. 17, 2016.

(51) Int. Cl. A61N 1/05

(52) U.S. Cl. CPC A61N 1/0563; A61N 1/368; A61N 1/0573; A61N 1/0592

(57) ABSTRACT

Implantation of a cardiac stimulus system into the mediastinum using the ITV. Superior, intercostal, and inferior access methods are discussed and disclosed. Superior access may be performed using the brachiocephalic vein to access the ITV, with access to the brachiocephalic vein achieved using subclavian vein, using standard visualization techniques. Inferior access may be accomplished inferior to the lower rib margin via the superior epigastric vein. Intercostal access may include creating an opening in an intercostal space between two ribs and advancing a needle using ultrasound guidance. Once in the ITV, access is then made into the mediastinum for placement of at least a portion of a lead of the cardiac stimulus system therein.

### CROSS REFERENCE TO RELATED APPLICATIONS

The present application claims the benefit of and priority to U.S. Provisional Patent Application Ser. No. 62/423,630, filed Nov. 17, 2016, titled ELECTRODE FOR SENSING, PACING, AND DEFIBRILLATION DEPLOYABLE IN THE MEDIASTINAL SPACE, the disclosure of which is incorporated herein by reference.

### BACKGROUND

The implantable defibrillator has been demonstrated to extend patient lives by treatment of potentially deadly arrhythmias. Over time, various efforts have been made to address complications associated with implantation of such devices. For example, early devices generally used epicardial patch electrodes implanted via thoracotomy, with attendant surgical risks and significant risks of failure of the epicardial patch electrodes and associated leads. The use of transvenous leads represented a major advance, avoiding the thoracotomy and improving reliability. However, lead failure remained a significant issue, as the lead attachment in the heart cause the lead to flex with each heartbeat. The advent of subcutaneous defibrillators allows avoidance of these lead failure issues, with leads implanted beneath the skin and over the ribcage of the patient and not subjected to the repeated flexing.

However, subcutaneous defibrillators require higher energy for defibrillation, causing the pulse generators for such systems to be larger than their transvenous predecessors, and both bradycardia pacing and anti-tachycardia pacing to avoid high voltage shock for certain conditions, is of limited utility as such pacing subcutaneously can be very uncomfortable for the patient. This has led to interest in further alternative locations for implantable defibrillators, and other medical devices such as the implantable pacemaker.

### OVERVIEW

The present inventors have recognized, among other things, that the internal thoracic vasculature including, in particular, the internal thoracic vein (ITV), sometimes also referred to as the internal mammary vein, presents an opportunity for an additional alternative implant location. A lead for an implantable cardiac device may be implanted into the mediastinum through one or both ITVs.

In a first example, a method of implanting a lead for use in a cardiac stimulus system in a patient, the lead having at least one electrode thereon may comprise accessing the mediastinum by entering the internal thoracic vein (ITV) and then exiting the ITV to enter the mediastinum, creating a recess in the mediastinum, and inserting the lead into the recess created in the mediastinum to a desired location relative to the heart of a patient.

In another example, a method of treating a patient may comprise delivering therapy between a first electrode disposed on a lead which is placed in a recess created in a mediastinum of a patient through an ITV, and at least a second electrode.

In another example, a method of implanting a lead for use in a cardiac stimulus system in a patient, the lead having at least one electrode thereon may comprise inserting a distal end of a lead into in a recess created in the mediastinum adjacent to the ITV, advancing the lead to a desired location relative to the heart of a patient, and securing the lead in place.

In another example, an implantable cardiac stimulus device may comprise a lead and an implantable canister for coupling to the lead. The implantable canister may house operational circuitry configured to deliver output therapy in the form of at least one of bradycardia pacing, anti-tachycardia pacing, cardiac resynchronization therapy, or defibrillation, according to any of the examples herein.

This overview is intended to provide an introduction to the subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates the thoracic anatomy including placement of the internal thoracic veins (ITVs);

FIG. 2 shows the torso in a section view to highlight the location of the ITVs and arteries;

FIGS. 3A-3B show the ITVs and linked vasculature in isolation;

FIGS. 4-5 show superior access to and implantation of a lead in the left ITV;

FIG. 6A shows in close view a location inferior to the lower rib margin where the ITV may be accessed inferiorly via the superior epigastric vein;

FIG. 6B illustrates intercostal access locations usable for superior or inferior access;

FIG. 7 shows implantation from an inferior position in a right ITV;

FIG. 8A shows implantation from an inferior position in both ITVs;

FIG. 8B shows an illustrative lead that may be used in the implantation configuration of FIG. 8A;

FIG. 9 shows implantation using an intercostal access to the right ITV;

FIGS. 10-19 illustrate various lead designs;

FIG. 20 is a block flow diagram for an illustrative method;

FIG. 21 is a lateral view of a method of implanting a lead using the ITV;

FIGS. 22-25 illustrate a close lateral view of a method of implanting a lead using the ITV;

FIGS. 26-29 illustrate a close lateral view of another method of implanting a lead using the ITV;

FIG. 30 shows an illustrative electrode for use with an implantable cardiac rhythm management system;

FIGS. 31A-31B show another illustrative electrode for use with an implantable cardiac rhythm management system;

FIGS. 32A-32B show another illustrative electrode for use with an implantable cardiac rhythm management system;

FIG. 33 shows another illustrative electrode for use with an implantable cardiac rhythm management system;

FIG. 34 shows another illustrative electrode for use with an implantable cardiac rhythm management system;

FIGS. 35A-35E show another illustrative electrode for use with an implantable cardiac rhythm management system;

FIGS. 36A-36B show another illustrative electrode for use with an implantable cardiac rhythm management system;

FIG. 37 is a lateral view of devices using the ITV concomitant with an LCP;

FIG. 38 shows superior access to and implantation of a lead in the mediastinum adjacent to the left ITV;

FIG. 39 shows inferior access to and implantation of a lead in the mediastinum adjacent to the right ITV;

FIG. 40 shows implantation in the mediastinum using an intercostal access to the right ITV; and

FIG. 41 is a block flow diagram for an illustrative method.

### DETAILED DESCRIPTION

The S-ICD System from Boston Scientific provides benefits to the patient including the preservation of transvenous anatomy and avoidance of intracardiac leads, which may fracture and/or may serve as conduits for infection to reach the heart, and can occlude blood vessels going into the heart, making later placement of leads or other devices in the heart more difficult. Some examples and discussion of subcutaneous lead implantation may be found in U.S. Pat. No. 8,157,813, titled APPARATUS AND METHOD FOR SUBCUTANEOUS ELECTRODE INSERTION, and US PG Publication No. 20120029335, titled SUBCUTANEOUS LEADS AND METHODS OF IMPLANT AND EXPLANT, the disclosures of which are incorporated herein by reference. Additional subcutaneous placements are discussed in U.S. Pat. No. 6,721,597, titled SUBCUTANEOUS ONLY IMPLANTABLE CARDIOVERTER DEFIBRILLATOR AND OPTIONAL PACER, and the above mentioned U.S. Pat. No. 7,149,575, the disclosures of which are incorporated herein by reference.

While many patients can be well treated with the S-ICD System, there continue to be limitations. Increased energy requirements of the S-ICD System, perceived difficulty with providing chronic bradycardia pacing, and unavailability of anti-tachycardia pacing to terminate select fast tachycardias, have created interest in alternative defibrillator and/or pacemaker placement techniques. One proposal has included a substernal placement, with a lead extending beneath the sternum from a position inferior to the lower rib margin, such as in US PG Patent Application Pub. No. 20170021159, titled SUBSTERNAL PLACEMENT OF A PACING OR DEFIBRILLATING ELECTRODE, the disclosure of which is incorporated herein by reference. Proposals for a substernal device have been referred to as extravascular, insofar as the lead does not enter or reside in the vasculature. Such devices are distinct from early generation epicardial devices in that the lead and electrode would not touch the heart or enter or be secured to the pericardium.

The present inventors have identified still a further alternative. In human anatomy, the internal thoracic vein (ITV), which may also be referred to as the internal mammary vein, is a vessel that drains the chest wall and breasts. There are both left and right internal thoracic veins on either side of the sternum, beneath the ribs. The ITV arises from the superior epigastric vein, accompanies the internal thoracic artery along its course and terminates in the brachiocephalic vein. The inventors have recognized that the ITV may make a suitable location for placement of a cardiac stimulus lead or may be used to place a cardiac stimulus lead in the mediastinum. While much of the following disclosure focuses on the use of the ITV, many of these concepts could also be applied to the internal thoracic arteries, which may sometimes be referenced as the internal mammary arteries.

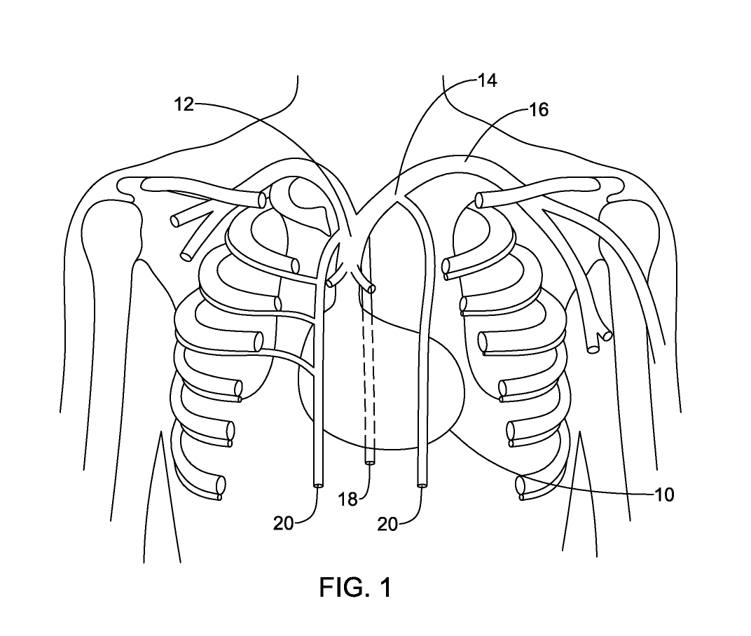

FIG. 1 illustrates the thoracic anatomy including location of the internal thoracic veins (ITVs). An outline of the heart is shown at 10, with the superior vena cava (SVC) shown at 12. The brachiocephalic veins 14 couple to the SVC and extend past various cephalic branches to the subclavian vein 16. The azygos vein is shown at 18, and the right and left ITV are shown at 20.

Certain literature in the field of implantable pacemakers or defibrillators has noted the possibility of the using the azygos vein 18 to implant a lead and electrode to stimulate the vagus nerve (see, for example, U.S. Pat. No. 8,005,543, the disclosure of which is incorporated herein by reference), or as an adjunct to defibrillator function (see Cesario et al., “Azygos vein lead implantation: a novel adjunctive technique for implantable cardioverter defibrillator placement,” J. Cardiovasc. Electrophysiol., 2004, 15:780-783). However, such proposals have not found widespread acceptance. However, it does not appear that the ITVs 20 have been proposed.

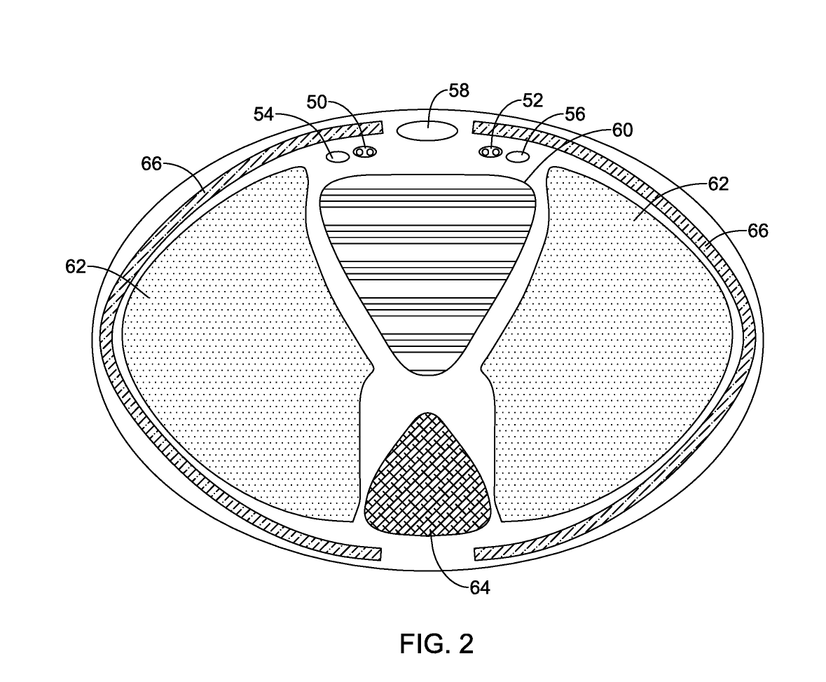

FIG. 2 shows the torso in a section view to highlight the location of the ITVs and internal thoracic arteries. More particularly, in the example, the left and right ITV are shown at 50, 52, running parallel to and more central of the internal thoracic arteries 54, 56, on either side of the sternum 58. The heart is shown at 60, with the lungs at 62 and spinal column at 64. The ITV 50, 52 lie beneath the ribs 66 but outside and separate from the pleurae of lungs 62. As used herein, the “ITV” is the name applied for the vein while it runs beneath the chest, that is, superior to the lower margin of the ribs. Inferior to the lower margin of the ribs, the blood vessel continues as the superior epigastric vein. The relatively superficial position makes the ITV 50, 52 accessible percutaneously inferior to the rib margin or through intercostal spaces between ribs 66 as further discussed below. Access to the ITV from an access point inferior to the lower rib margin may be described as accessing the ITV via the superior epigastric vein. Also shown in some examples below are methods to access to the ITV via the superior vasculature, including the brachiocephalic vein.

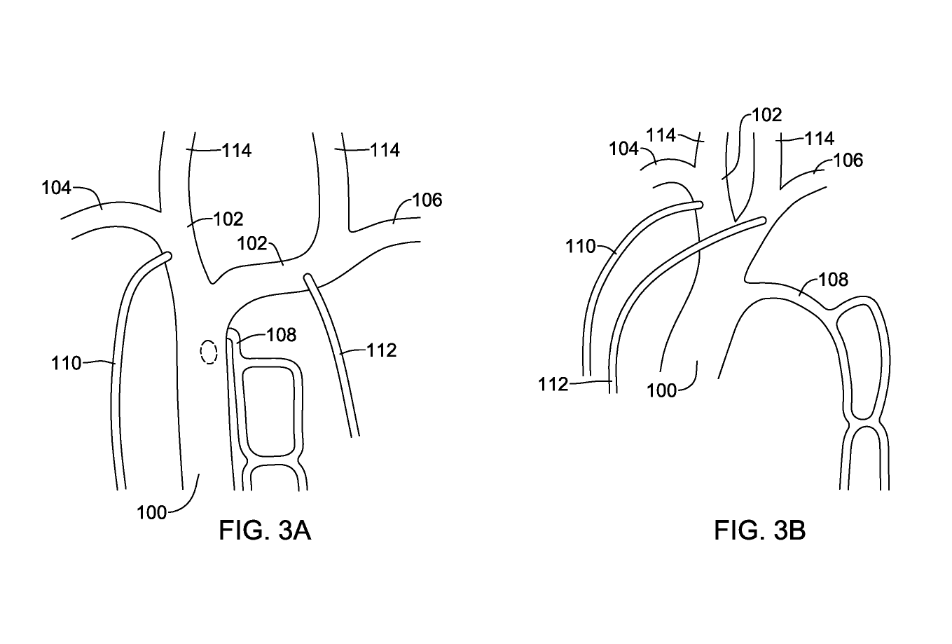

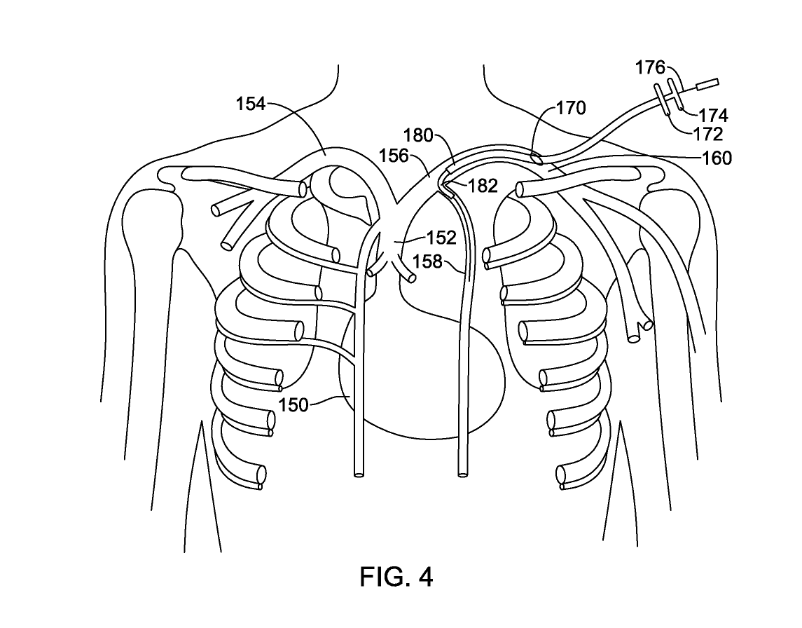

FIGS. 3A-3B show the ITV and linked vasculature in isolation. FIG. 3A is an anterior view of selected portions of the venous structure of the upper torso, and FIG. 3B is a lateral view of the same. The SVC is shown at 100, with the brachiocephalic veins 102 splitting at the upper end of the SVC. The right subclavian vein is at 104, and the left subclavian vein is at 106. The azygos vein is include in the illustration at 108, extending off the posterior of the SVC, and runs inferiorly posterior of the heart as can be understood from the lateral view of FIG. 3B. The right and left ITV are shown at 110, 112. These each branch off at a location that is considered part of the brachiocephalic veins 102. The internal jugular veins are also shown at 114.

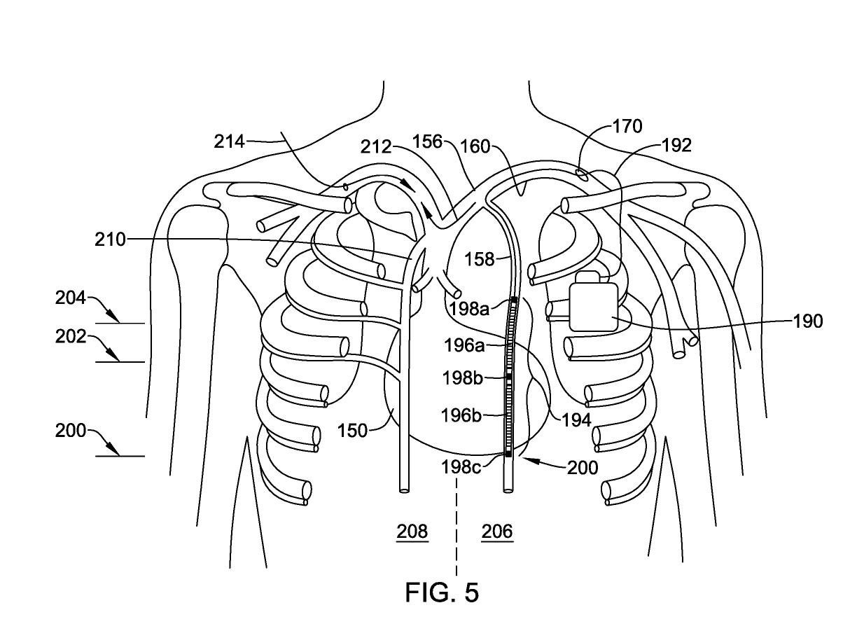

FIGS. 4-5 show superior access to and implantation of a lead in the ITV. Starting with FIG. 4, the heart is shown at 150 with the SVC at 152 and the brachiocephalic vein right branch at 154 and left branch at 156. Access to the subclavian vein 160 is shown at 170 using standard access techniques known in the art for implanting traditional transvenous pacemakers and defibrillators. For example, the Seldinger technique may be used by creating a puncture with a hollow needle or trocar, for example under ultrasound guidance, introducing a guidewire through the needle, removing the needle, and then inserting an introducer sheath 172, which may have a valve at its proximal end, over the guidewire. Other venipuncture or cutdown techniques may be used instead. Other vessels may be accessed instead of the subclavian vein using similar techniques including, for example, the jugular, cephalic, or axillary veins.

Into the access at 170, an introducer sheath 172 is inserted and advanced to a location to place its distal tip 180 near the ostium of the left ITV 158. Contrast injection may be useful to visualize the ITV structures and the ostia of the ITVs. A guide catheter 174 and guidewire 176 are then introduced through the introducer sheath 172. In other examples, a shorter introducer sheath may be used, with the guide catheter 174 used to traverse the distance to the relevant ostium.

The guidewire may be the same as used in gaining initial access 170 (if one is used to gain access 170), or may be a different guidewire. In an example, the guidewire 176 is preloaded in the guide catheter and both are introduced at the same time until the guide catheter 174 is at a desired location relative to the ostium of the selected ITV. The guidewire 176, which may be deflectable or steerable, can then be used to enter the left ITV 158 through the ostium thereof, passing down into the left ITV 158. The guide catheter 174 can then traverse over the guidewire and through the ostium and into the left ITV 158.

A device passing into the ITV from a superior position will need to pass through the valves of the ITV in a direction counter to their natural tendency (the veins prevent blood from flowing inferiorly). For an example where the guidewire passes unsupported by a guide catheter into the ITV from a superior position, the guidewire may preferably be stiff. In some examples, at least two guidewires may be used, a first more flexible and steerable guidewire to obtain initial access via the ostium of the ITV, and a second, stiffer guidewire that is sufficiently pushable to allow passage through the valves in the ITV.

In some examples, the guide catheter 174 is introduced first and the guidewire 176 is introduced next. For example, a steerable or curved guide catheter 174 may traverse the introducer sheath 172 to its distal end 180 and then, using steering of the guide catheter or a precurved structure of the guide catheter, would then turn as shown at 182 to enter the left ITV 158. The guidewire 176 may be introduced through the guide catheter 174. In another example, a guidewire 176 may be omitted.

FIG. 5 shows implantation of an implantable cardiac stimulus system. The system includes an implantable pulse generator 190 which may be placed in the subclavicular location shown (or any other suitable position, as desired). A lead 192 passes into the venous access point 170 into the subclavian vein 160 and to the brachiocephalic vein 156. The lead then enters the left ITV 158. For such an introduction, in one example, the guide catheter 174 (FIG. 4) can be used to direct the lead 192 through the ostium of the chosen ITV, with or without use of a guidewire 176 (FIG. 4).

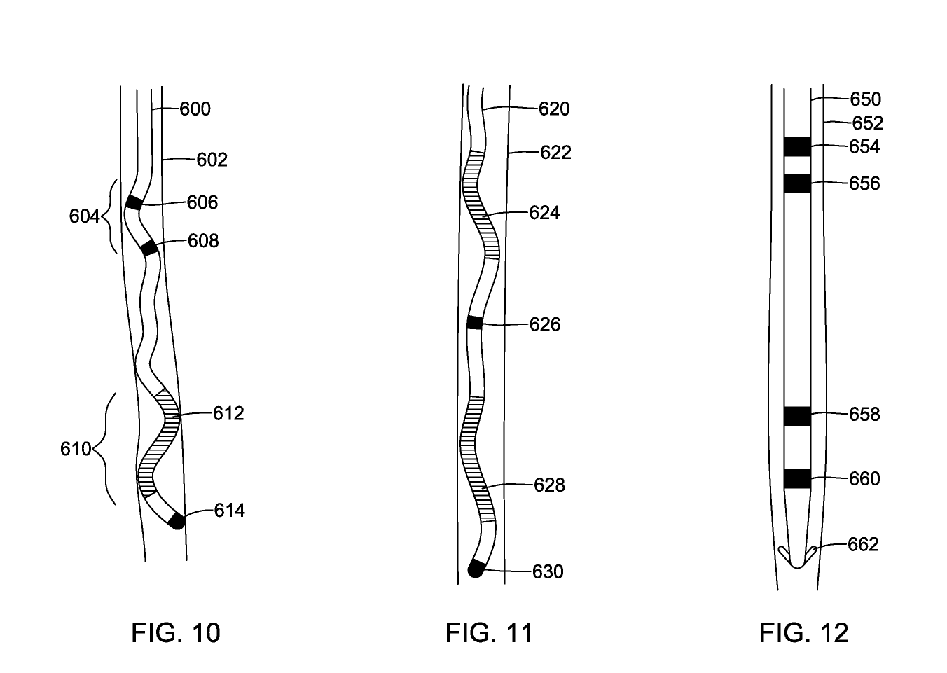

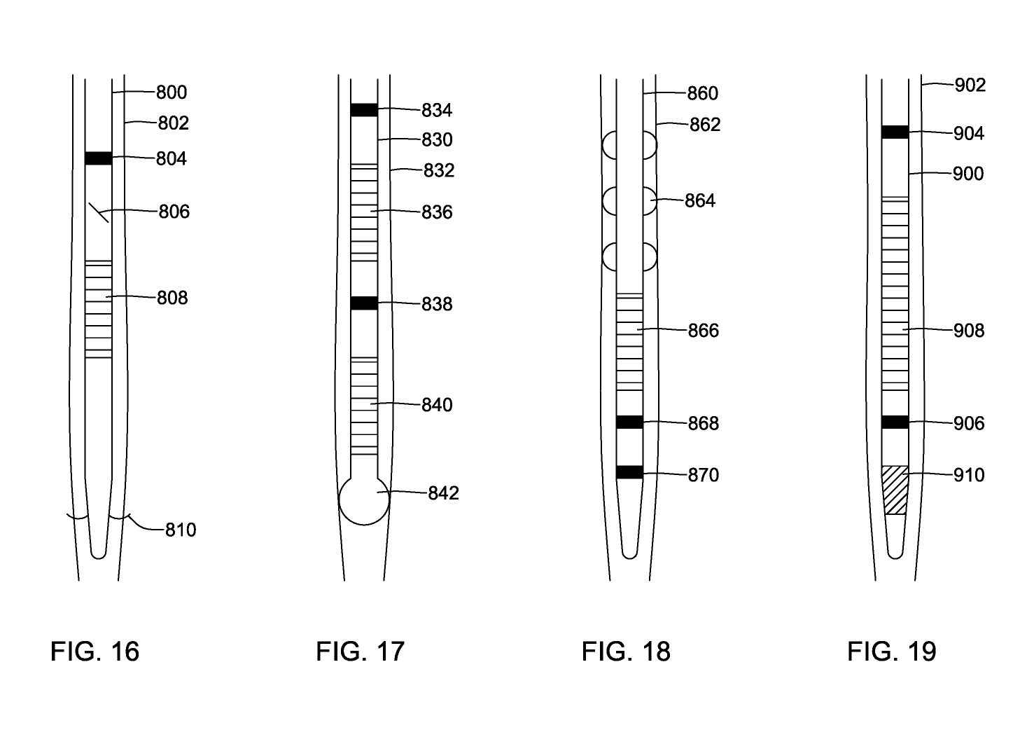

In some examples, a flexible lead is used having a lumen therein to receive a guidewire or stylet to enhance pushability through the valves of the ITV 158. In another example, a flexible lead may be introduced with the support of the guide catheter 174 during advancement. In this latter example, the guide catheter 174 may receive the lead 192 through a guide catheter lumen that serves to retain a fixation apparatus or shape for the flexible lead, such as a 2-dimensional or 3-dimensional curvature (see FIGS. 10-11), tines (see FIG. 12), an expandable member (see FIG. 15), or hooks or a side-extending engagement structure (see FIG. 16).

In another alternative, the guide catheter 174 and guidewire 176 may be omitted by providing a lead with a flexible or steerable structure, and/or a lead configured for implantation using a steerable stylet. For example, a lead may be configured to be implanted using a steerable stylet in a lumen thereof, with the initial placement into the ostium of the left ITV 158 (or right ITV 210, if desired) at the distal end of the introducer sheath 172, possibly using contrast visualization, if desired. Once initial access is achieved, simply pushing the stylet should be sufficient to implant the lead to a desired level in the ITV. The stylet may have a secondary function of preventing an anchoring structure of the lead from assuming an anchoring shape or releasing an anchoring tine, hook, expandable member, stent or other device.

In the example, the lead 192 includes a multi-electrode distal structure as shown at 194. The structure includes a proximal coil 196A separate from a distal coil 196B. The coils 196A/B and canister 190 may serve as therapy delivery electrodes. As such there may be multiple therapy vectors such as between coil 196A and coil 196B, between either of coils 196A and 196B and the canister 190, or between a combination of two of the three therapy electrodes 196A, 196B and canister 190, and the third such electrode, such as by linking coils 196A and 196B in common as the anode or cathode relative to the canister 190.

A plurality of ring electrodes may be provided as shown at 198A, 198B, and 198C. Electrode 198C may also or instead be a tip electrode. Electrodes 198A/B/C may serve as sensing electrodes. The coils 196A, 196B may also serve as sensing electrodes. These various electrodes may be used for sensing cardiac signals in various combinations using, for example, methods and circuitry discussed in U.S. Pat. No. 7,783,340, titled SYSTEMS AND METHODS FOR SENSING VECTOR SELECTION IN AN IMPLANTABLE MEDICAL DEVICE USING A POLYNOMIAL APPROACH, and U.S. Pat. No. 8,483,843, SENSING VECTOR SELECTION IN A CARDIAC STIMULUS DEVICE WITH POSTURAL ASSESSMENT, and/or US PG Patent Application Pub. Nos. 20170112399, 20170113040, 20170113050, and 20170113053, the disclosures of which are incorporated herein by reference.

In addition, one or more of the ring or tip electrodes 198A, 198B, 198C may be used for therapy delivery. In an example, defibrillation therapy may use coils 196A, 196B coupled in common as the opposing pole to the canister 190, while pacing therapy may use coils 196A and 198B as opposing electrodes for post-shock pacing therapy, with a still different combination of electrodes used to provide ventricular pacing therapy for example by pacing between coil 196B and tip electrode 198C.

Line 202 is provided, illustratively, to separate the atria and ventricles. The lead 192 may be placed as shown such that the proximal coil 196A is about level with the atria, and distal coil 196B is about level with the ventricles, if desired. In some examples fewer or different electrodes may be provided on the lead 192 such as by excluding one or the other of the proximal coil 196A or distal coil 196B. Various designs are also shown herein.

Line 204 is provided to indicate the top of the heart, with the apex or bottom of the heart marked at 200. In some examples, one or more electrodes on the lead 192 are provided at or inferior to the apex 200, or at or superior to the top 204 of the heart. In the example shown, on the other hand, the electrodes are located generally between the apex 200 and top 204 of the heart.

The illustration shown in FIG. 5 places the lead on the left side 206 of the patient. In other examples, the right side 208 of the patient may instead or in addition be accessed, including the right ITV 210. Access to the right ITV 210 may be achieved by advancing a guide catheter and/or guidewire from the left subclavian access 170 as shown by arrow 212 across to the ostium of the right ITV 210.

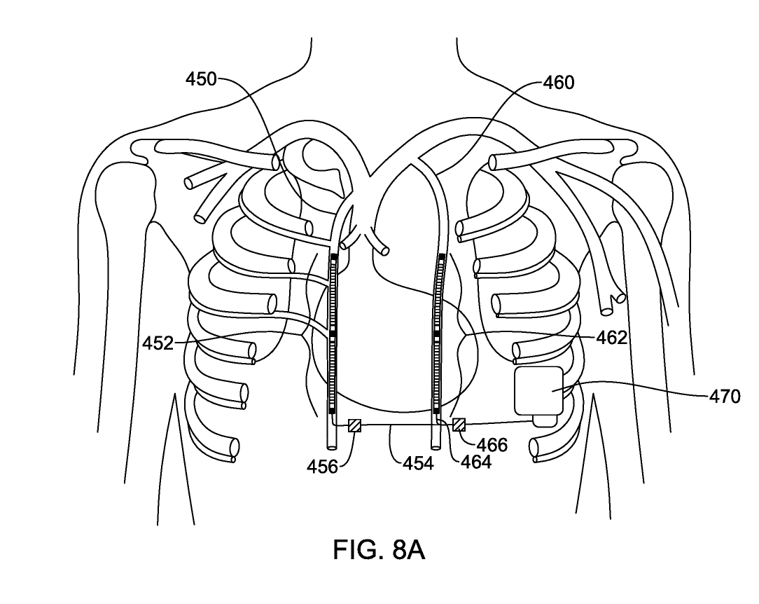

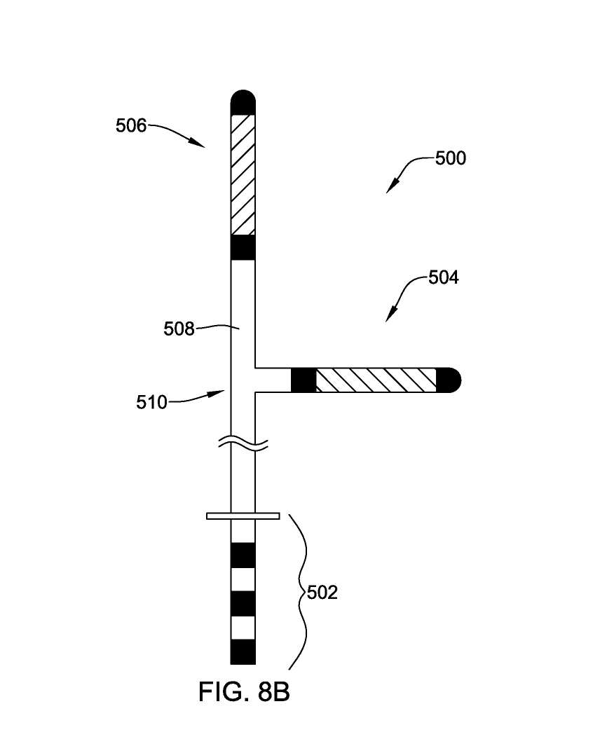

Alternatively, access to the right ITV may be achieved as shown at arrow 214 by entering the right subclavian vein in a mirror image procedure of that shown in FIG. 4. In some examples, each of the left and right ITV 158, 210 may receive a lead 192. The lead 192 may be split (as shown relative to an inferior access route in FIG. 8B), a yoke may be provided near the canister 190 to join two leads together, or a header on the canister 190 may be configured to receive more than one lead 192, if desired, to provide leads in each of the left and right ITV 158, 210. If two leads are provided, use may be similar to that explained relative to FIG. 8A, except insofar as the leads may be implanted from the superior blood vessels as shown in FIG. 5. For example, pacing between right and left side lead placements may be performed to target specific chambers or chamber combinations, or sensing may be performed using one pair of electrodes with therapy delivery using a different pair of electrodes to achieve resynchronization or other desirable effect.

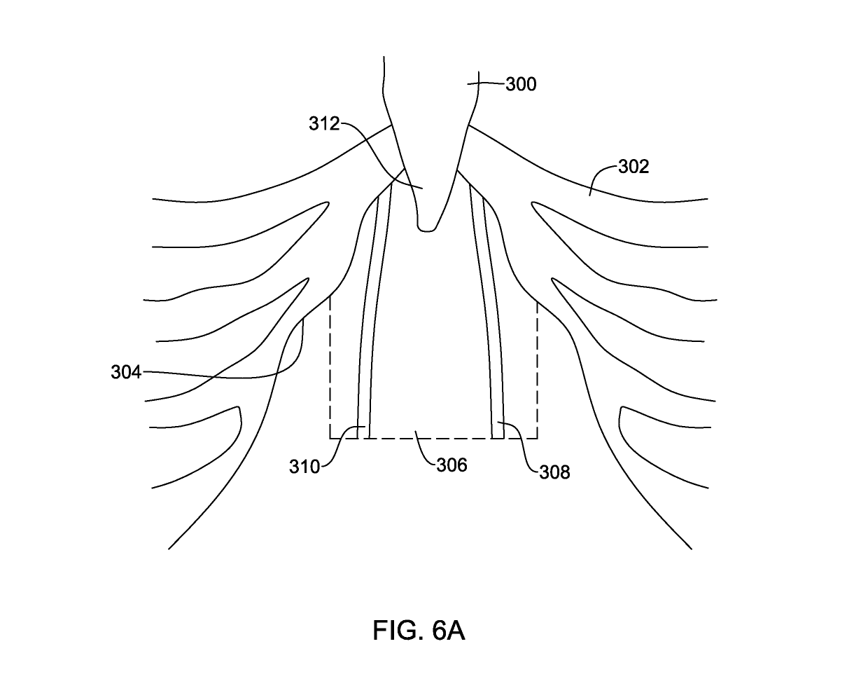

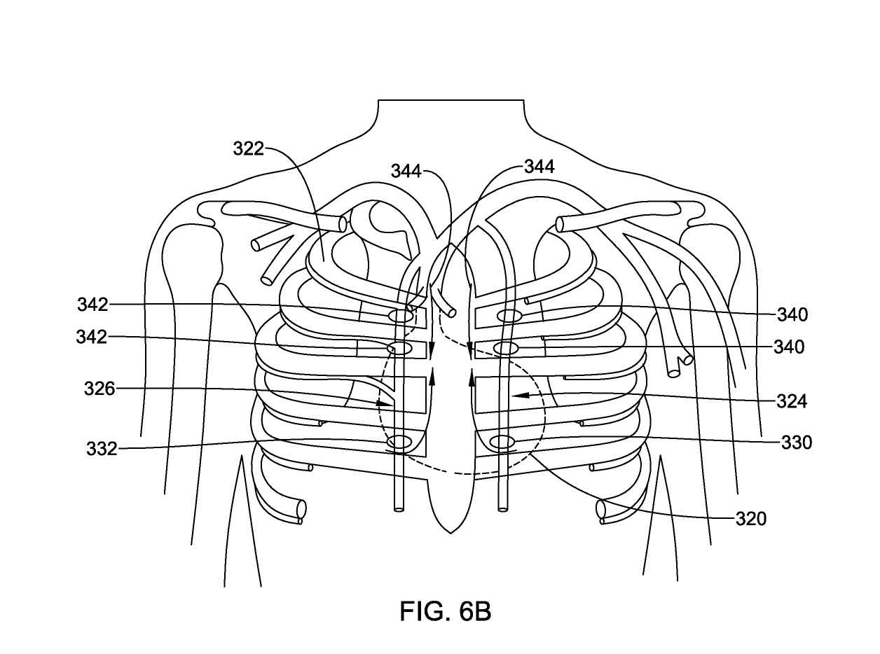

FIG. 6A shows in close view a location inferior to the lower rib margin where the ITV may be accessed inferiorly. This region may be referred to as the inferior thoracic aperture. The patient anatomy is shown in part including the sternum 300 and ribs 302, with the lower rib margin at 304.

A cutout area is shown at 306 in order to illustrate the approximate location for accessing the right or left ITV using the superior epigastric veins. The left superior epigastric vein is shown at 308, and the right superior epigastric vein is shown at 310. In order to access either vein 308, 310, a physician may palpate for the xiphoid process 312 and then use ultrasound guided access to obtain needle entry into the desired vein 308, 310 on the desired side of the xiphoid 312. This inferior approach preserves the upper thoracic vasculature in the event that the patient later needs a traditional transvenous, intracardiac system, or for use in other procedures. Such access may also reduce the potential for lead fracture such as that caused by subclavian crush. Once access to a selected superior epigastric vein 308, 310 is achieved, the vessel can be traversed in a superior direction to place the lead at a desired level by entering the corresponding ITV.

The access may generally resemble the well-known Seldinger technique, with an initial needle puncture using a hollow needle or trocar. A guidewire is passed through the hollow needle or trocar, which can then be removed. An introducer sheath, typically having a dilator therein and a valve at a proximal end thereof, is then inserted over the guidewire and into the desired blood vessel. The dilator and/or guidewire can then be removed, leaving in place the valved introducer sheath to allow introduction of interventional devices and/or a lead therethrough. At the conclusion of the lead implantation procedure, a sealing device such as a suture sleeve can be placed to seal the puncture site to the implantable lead left therein. The aim may be to access the ITV or superior epigastric vein at or near the 7^{th} rib margin in a window adjacent to the xiphoid process that may be described as a paraxiphoid window.

In another example, a cut-down technique may be used to access the desired vein 308, 310 by incision through the skin. Next, possibly after visual confirmation the desired vessel is accessed, incision into the selected vein can be made. In another example, anatomical landmarks such as the rib margin and/or infrasternal angle may be used to facilitate venipuncture into the desired vein 308, 310.

In animal testing the present inventors have determined that access to the ITV can be achieved with little difficulty to facilitate lead placement by accessing the superior epigastric vein in the region adjacent and inferior to the lower rib margin. However it is recognized that the human anatomy will be different from that of the tested animal (porcine model), and may further vary with the particular body characteristics of a given patient including, for example, any venous abnormality, scarring in the area (such as related to any prior sternotomy or the like) as well as the body habitus (overweight or underweight patients).

The musculophrenic vein (not shown) runs along the lower rib margin 304 and may instead, or also, be accessed in a manner that will be termed, for purposes herein, as an inferior access location as it would be inferior to the lowest rib. The musculophrenic vein and superior epigastric vein come together at the lowest end of the ITV. The musculophrenic vein may be accessed using similar methods as for the superior epigastric vein such as by ultrasound-guided Seldinger technique. Due to its adjacency to a bony structure (the costal margin at 304), the musculophrenic vein may be useful as its access may be simpler than that of the superior epigastric vein, as the position can be readily ascertained. Further details on use of the musculophrenic vein for ITV access can be found in U.S. patent application Ser. No. 15/667,167, titled IMPLANTATION OF AN ACTIVE MEDICAL DEVICE USING THE INTERNAL THORACIC VASCULATURE, the disclosure of which is incorporated herein by reference.

FIG. 6B illustrates some intercostal access locations usable for superior or inferior access. The Figure shows the heart at 320 beneath the ribcage 322. The right and left ITV are shown at 324 and 326. Any intercostal space overlying either of the right and left ITV may be a suitable point of entry, however, more superior or inferior positions may be preferred to allow passage of the distal end of a lead along a significant region of the ventricles and atria by passing in a single direction.

In the example shown, illustrative intercostal access locations are shown at relatively inferior positions 330, 332, and more superior positions 340, 342. In either case, access may be had using ultrasound guided needle insertion. Again, the access method may resemble the Seldinger technique, though in this case the muscle in the intercostal space would first be traversed. A needle may be used to establish puncture using ultrasound guidance, with a guidewire passed therethrough. Once the puncture is made and the guidewire is in the desired blood vessel, the needle is removed, keeping the guidewire in place, and an appropriately sized introducer sheath (optionally including a dilator) is placed over the guidewire.

The alternative in FIG. 6B allows access from either superior or inferior positions while preserving the upper thoracic vasculature. Such an access position may be labeled a parasternal access position. An advantage over the approach of FIG. 6A is that the use of a suture sleeve attachment with FIG. 6B would occur on the fascia over the ribcage near the intercostal access point, making suture sleeve use easier and avoiding movement between the point of venous system entry and the point of fixation. On the other hand, a user may be more comfortable accessing the veins at a location where the ribs and intercostal muscles do not interfere; thus, each of the various approaches herein has advantages and disadvantages relative to one another.

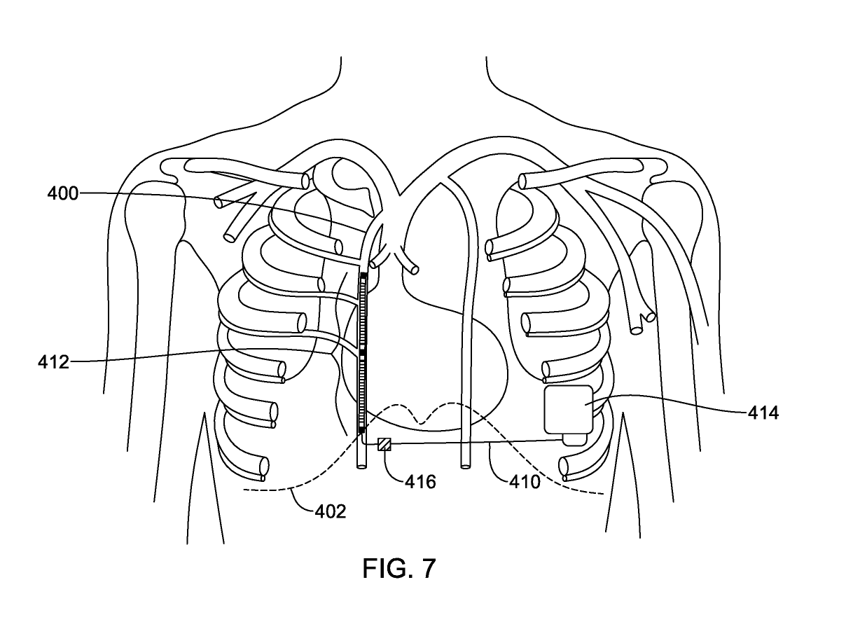

FIG. 7 shows implantation from an inferior position in an ITV. In this example, the right ITV 400 has been accessed by introduction through the superior epigastric vein from a location inferior to the rib margin 402. An implantable device has been placed including a lead 410 having a distal electrode structure 412 and a canister 414, with the canister 414 placed at approximately the left axilla. The canister 414 may be placed as desired, for example at the anterior axillary line, the midaxillary line, or in the posterior axillary line.

In the illustration, a suture sleeve is shown at 416 and is used to fixate the lead 410, for example, to the subcutaneous fascia. For placement, the right ITV 400 is accessed as described above, and a tunnel is established between the left axilla and the access location such as along a portion of the inframammary crease. The lead 410 may, in this case, be relatively stiff to assist in keeping it emplaced in the patient as shown, if desired. Various designs are shown herein for the lead as well, including tines, hooks, curvature or bias of the lead, and inflatable or expandable structures. In the example of FIG. 7, a left axillary canister location is shown; a right sided, pectoral or subclavicular left or right position may be used instead, in combination with the right ITV placement 400 or, alternatively a left ITV placement.

During implantation, a sheath may be provided over the lead 410, or at least a portion thereof, to retain or restrain a fixation apparatus or shape for the flexible lead, such as a 2 or 3 dimensional curvature (see FIGS. 10-11), tines (see FIG. 12), an expandable member (see FIG. 15), or hooks or a side-extending engagement structure (see FIG. 16). A stylet may be placed through the lead 410, or a portion thereof, to retain a straight shape during implantation; upon removal of the stylet, a curvature (see FIGS. 10-11) may then be released for securing the lead 410 in place.

The lead 410 may include additional or different electrodes than those shown. For example, another coil electrode may be placed on a more proximal portion of the lead 410 to reside along the inframammary crease in a location between the canister 414 and the point of access into the superior epigastric vein. The additional coil at this location may be used for defibrillation or other therapy purposes, or for sensing. If desired, second or more leads may also be placed.

FIG. 8A shows implantation from an inferior position in both ITV. In this example, the right ITV 450 is shown with the electrode structure 452 on a distal end of a lead 454 disposed therein. A suture sleeve 456 secures the lead 454. The lead 454 includes a second branch that enters the left ITV 460 with a distal electrode structure 462 disposed therein. A second suture sleeve 466 optionally secures the lead 454 at a second location. A canister for the system is shown implanted in the left axilla. The point of access to each of the right and left superior epigastric veins, in order to enter the right and left ITV 450, 460, may be placed close to the xiphoid process at or near the paraxiphoid window, near the 7^{th} rib margin. More inferior access to the superior epigastric veins may be used if desired.

FIG. 8B shows an illustrative lead that may be used in the implantation configuration of FIG. 8A. The illustrative lead 500 includes a proximal plug structure shown at 502, with a split at 510, from which a shorter branch having an electrode structure 504 extends, and a longer branch 508 continuing in the axial direction to another electrode structure 506. The design is illustrative and not intended to be limiting. In another example, two separate leads may be used, rather than one integrated lead.

As shown, each electrode structure 504, 506 includes a coil electrode flanked with two sensing electrodes; other combinations of electrodes may be used. Each electrode may be electrically connected to a single contact on the plug 502 or, if desired, subsets of electrodes may be ganged together relative to a single contact on the plug 502. The distal portion may include a fixation apparatus or shape for the flexible lead, such as a 2 or 3 dimensional curve (see FIGS. 10-11), tines (see FIG. 12), an expandable member (see FIG. 15), or hooks or a side-extending engagement structure (see FIG. 16).

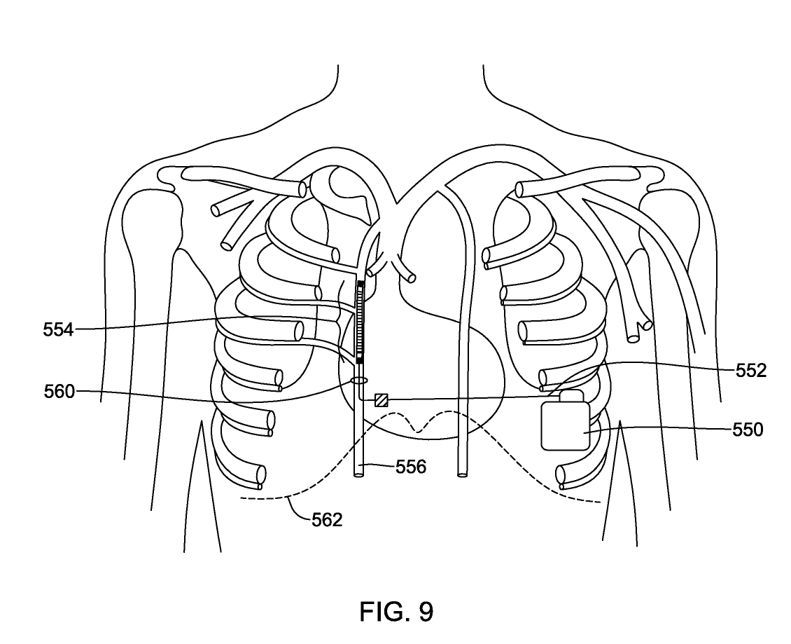

FIG. 9 shows implantation using an intercostal access to an ITV. In this example, an implantable system having an implantable pulse generator 550 and lead 552 with distal electrode structure 554 has been emplaced in a patient. The right ITV 556 is accessed using an intercostal access point at 560.

The intercostal access 560 may be achieved by inserting a needle, preferably under guidance such as by the use of an ultrasound guided needle, into a chosen intercostal space, preferably low on the ribcage and near the sternum, through the muscle of the intercostal space and into the right ITV 556. A guidewire can be passed through the needle and an introducer sheath passed over the guidewire after removal of the needle. Other techniques may be used instead, and other access points may be selected.

A suture sleeve may be used to secure the lead 552 over the ribcage as desired. The lead 552, as with all other implanted leads shown herein, may include a fixation structure such as bends or curves along its distal length, or tines, hooks or expandable members at its distal end to secure its position within the ITV 552.

FIGS. 10-18 illustrate various lead designs. These leads may be manufactured of any suitable material and by any suitable manner. For example, numerous polymers are known for lead manufacture. Internal longitudinal or lateral support members, such as braids, core wires, etc. may be provided. Extrusion or molding may be used. Internal conductors may be formed of any suitable material (stainless steel, titanium, gold, silver, or any other conductive material may be used) and may take any suitable form, such as simple wires, coated wires, braided or wound wires, drawn wires, and/or drawn filled tubes, or other structures. The leads may include on all or a portion thereof various coatings such as an anti-microbial coating to reduce the likelihood, severity, and/or progression of infection. Some illustrative lists for such design details follow later in the disclosure.

FIG. 10 shows an illustrative lead structure. A lead 600 is shown within a blood vessel 602, which may be an ITV. The lead may include ring electrodes illustrated at 606, 608, and a tip electrode 614, as well as a coil electrode at 612. Regions of curvature area shown at 604, and at 610. A single curvature may be provided instead. The curvature may be two-dimensional or three-dimensional. A two dimensional curvature may take the form, generally, of a zig-zag design, for example. Several embodiments may use a three dimensional curvature such as a pigtail or helix, for example.

In one example, the distal tip 614 is implanted inferior relative to the rest of the lead, such that the coil 612 is adjacent or level with the patient's ventricles. In another example, the distal tip is implanted superior relative to the rest of the lead, such that the coil 612 is adjacent or level with the patient's atria. In another example, the position of coil 612 is switched with the position of ring electrode 608, such that if implanted with the tip 614 superior relative to the rest of the lead, the tip 614 would be at about the level of the atria (or higher), while the coil 612 would be adjacent to or level with the ventricles.

FIG. 11 shows another example. A lead 620 is shown within a blood vessel 622, which may be an ITV. The lead may include ring electrode 626 and a tip electrode 630, as well as coil electrodes 624, 628. An additional ring electrode may be placed proximal of the coil electrode 624, as shown above in FIG. 5, if desired. With this example, the coils 624 may be spaced and positioned such that one is level with the ventricles and the other is level with the atria when implanted with the tip 630 either superior or inferior. As with FIG. 10, FIG. 11 shows that the lead has several areas of curvature.

In FIGS. 10 and 11, the curvature may be assumed by the lead in several ways. In an example, the lead includes a shape memory material and is generally straight and flexible until implanted in the body; after a few minutes to warm up, the shape memory material assumes the shape shown. In another example, a stylet is placed inside the lead during implantation to retain a generally straight shape, and the lead assumes the curved shape shown when the stylet is removed. In another example, an outer sheath is used to retain the lead until it is implanted with removal of the outer sheath allowing the lead to assume a desired shape. Combinations may be used as well; for example, a lead may include a shape memory portion or material or support structure, and may be implanted with the aid of a stylet and outer sheath to retain a low profile for implantation and then, once released by removal of the stylet and sheath, the shape memory material exerts forces to assume the shapes shown. Though not shown, curvature may be used for secure placement of any of the leads shown in FIGS. 12-18, if desired.

FIG. 12 shows another example. Here, a lead 650 is shown inside a blood vessel 652, which may be the ITV. First and second ring electrodes are shown at 654, 656, and third and fourth ring electrodes are shown at 658, 660. Tines for fixation are shown at 662. The ring electrodes may be placed such that if the tines 662 are superior relative to the rest of the lead, electrodes 658, 660 would be level with the atria, and electrodes 654, 656 would be level with the ventricles. This may facilitate separate atrial and ventricular sensing and/or pacing channels. A coil electrode may also be provided.

In one example, a lead as shown in FIG. 12 is implanted in the left ITV while a separate lead is implanted in the right ITV, with the right ITV comprising a defibrillation coil electrode, with an active canister defibrillator implanted in the left axilla. This approach would allow sensing (and optionally, pacing) directly over the heart using the ring electrodes 654, 656, 658, 660, with defibrillation delivered across the majority of the myocardium between the right-sided coil electrode and the left sided canister.

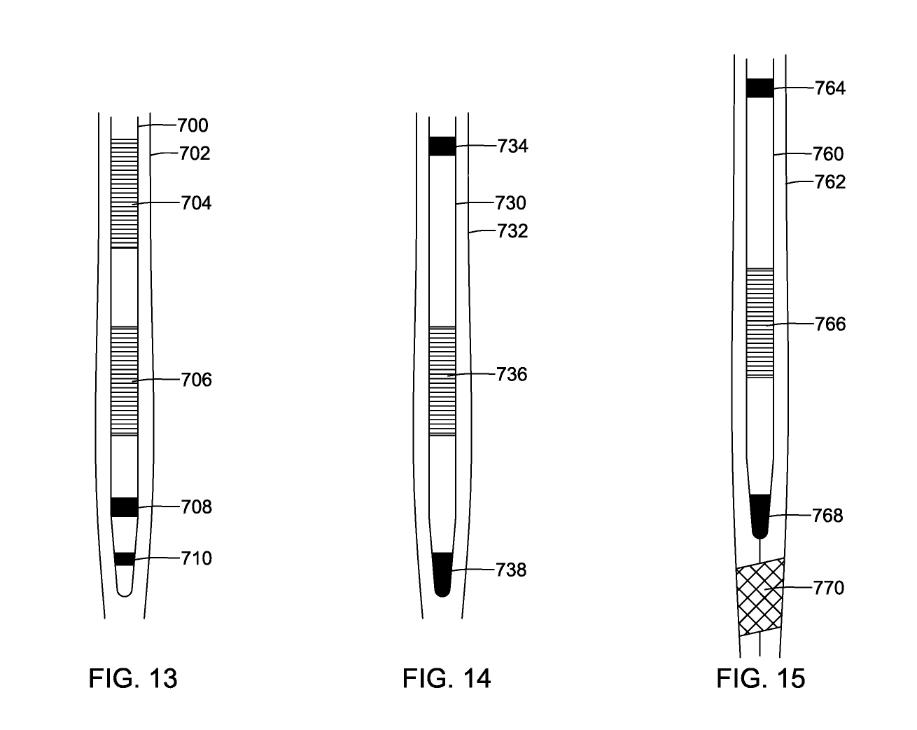

FIG. 13 shows another example. Here a lead 700 is implanted in a blood vessel 702 which may be an ITV. A first coil is shown at 704 and a second coil is shown at 706, with two distally located ring electrodes. If desired, the lead may taper as shown, though a fully cylindrical lead may be used instead. The taper may be useful during implantation to facilitate easier access through venous valves, particularly for insertions from superior to inferior, where the direction of insertion is counter to blood flow and hence valve structure. Curves or tines may be added, as well as other fixation features noted herein.

FIG. 14 shows another example. In this example, a lead 730 is shown inside of a blood vessel 732 which may be an ITV. A proximal ring electrode is shown at 734 and a coil at 736, with a distal tip electrode at 738. Curvature or tines may be added, as well as other fixation features noted herein.

FIG. 15 shows another example. Here, the lead is much as in FIG. 14, with lead 760 shown inside a blood vessel 762 which may be a ITV, and with a proximal ring electrode 764, coil electrode 766, and distal tip electrode 768. However, now, an expandable member, such as a stent 770 is shown distal to the distal tip electrode 768. For example, a self-expanding stent 770 may be provided and carried within the distal tip electrode 768 until a desired position is reached for the stent 770. Such positioning may be determined using, for example, fluoroscopy. The proximal end of the lead may include a release mechanism, such as a control wire that can be advanced relative to the lead body, to push the stent 770 beyond the distal tip electrode 768 where it can then release. Self-expanding stents are well known in the art and may include, for example, spring-like structures. The stent 770 may include coatings designed to prevent thrombus from forming thereon and/or to encourage angiogenesis to best engage the venous wall. For removal, the connection to the stent 770 may be cut, for example, to leave the stent 770 in place as the rest of the lead is removed. Optionally the stent may be later removed using, for example, a stent retriever.

FIG. 16 shows another example. Here, a lead 800 is shown in a blood vessel 802 which may be an ITV. A proximal coil electrode is shown at 804. Distal of the proximal coil electrode (though any suitable location, more proximal or more distal, may be chosen), a side-engaging member is shown at 806. For example, engaging member 806 may be an arm, coil, hook, or tine that expands outward when actuated from the proximal end of the lead. Once the lead is in a desired position, engaging member 806 may be actuated to secure the lead in place.

The lead 800 is also shown with a coil electrode at 808. Finally, at the distal tip of the lead, a plurality of hooks are shown for engaging the walls of the blood vessel 802. The engaging member 806 or hooks 810 may be coated as desired for anti-thrombogenic or pro-angiogenic reasons, for example.

FIG. 17 shows another example. Here, a lead 830 is shown inside of a blood vessel 832 which may be an ITV. A plurality of electrodes are shown including a ring electrode 834, coil electrode 836, ring electrode 838, and coil electrode 840. At the distal end of the lead is an expandable member, such as a balloon, which may be inflated to secure the lead in place. It should be noted that the ITV is a blood vessel which, if occluded, will not necessarily cause harm to the patient as contralateral accommodation occurs readily. The balloon 842 may be expanded using inflation pressure, for example. A compliant or non-complaint material may be used the balloon. Rather than a balloon, an expandable sponge-type member that increases in volume once sufficiently wetted may be used instead.

FIG. 18 shows another example. In this example, the lead 860 is shown in a blood vessel 862 which may be an ITV. This example includes a plurality of lobes 864 which hold the lead 860 in place inside the blood vessel 862. For example, the lobes may self-expand on removal of an outer delivery sheath or catheter, or the lobes may be expanded by movement of an outer shell of the lead relative to an inner shell. A coil electrode is shown at 866 and ring electrodes are shown at 868, 870.

FIG. 19 shows another example. A lead 900 is shown within a blood vessel 902, which may be an ITV. The lead may include ring electrodes illustrated at 904, 906, a coil electrode 908, and a motion detector, sound sensor, and/or accelerometer 910. If so provided, an accelerometer 910 may provide a clinician and/or the canister with heart motion data as well as heart sound data (e.g., the S3 heart sound). Either or both the heart motion data and the heart sound data may be used to provide heart failure status information.

The examples of FIGS. 10-19 are merely illustrative. Some examples may omit any fixation on the portion of the lead that extends into the blood vessel, and may instead rely on fixation using a suture sleeve subcutaneously placed as shown in certain of the above examples. In some examples, a relatively stiff lead may be used, as repeated flexion is not necessary when implanted in the ITV in the same manner as is the case inside the heart. A stiff lead is believed to be less likely to migrate.

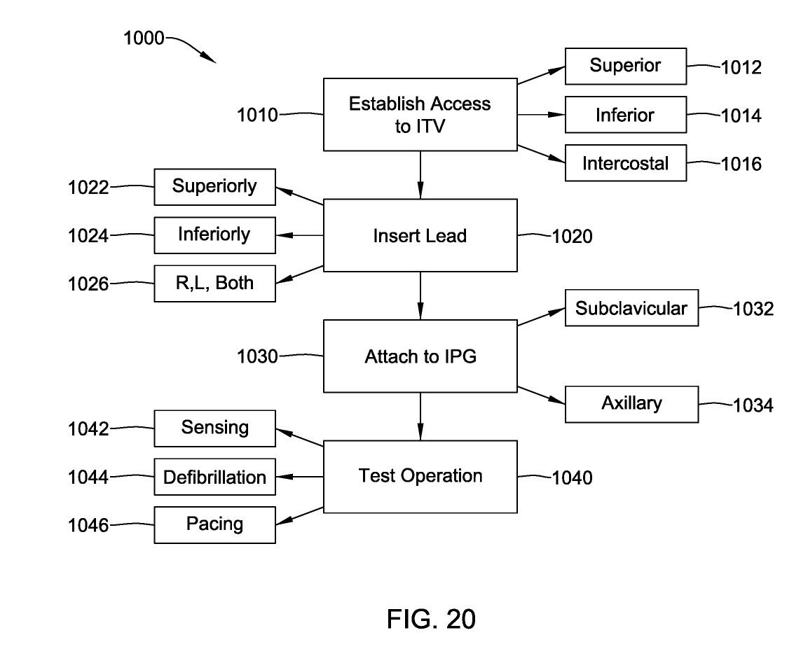

FIG. 20 is a block flow diagram for an illustrative method for providing a cardiac stimulus system to a patient. As shown at 1000, the method comprises establishing access to the ITV 1010, inserting a lead in the ITV 1020, attaching an IPG to the lead 1030, and performing test operations 1040.

For example, establishing access to the ITV 1010 may include accessing from a superior position 1012 such as by entering the subclavian vein and passing through the ostium of the ITV in the brachiocephalic vein. In another example, establishing access to the ITV 1010 may include accessing from an inferior position 1014 such as by entering the superior epigastric vein and passing superiorly therefrom into the ITV. In some examples, access via locations 1012, and 1014 may include accessing via a second blood vessel such as by accessing superiorly 1012 by way of the subclavicular vein and brachiocephalic vein, or accessing inferiorly 1014 through the superior epigastric vein. In still another example, establishing access to the ITV may include accessing in an intercostal space 1016 such as by penetrating an intercostal space and entering the ITV using a Seldinger technique.

In an example, inserting a lead 1020 may include insertion superiorly 1022, such as by starting in an inferior position 1012 inferior to the lower rib margin or intercostally 1016 from an inferior intercostal location, and advancing the lead in a superior direction. For another example, inserting a lead 1020 may include insertion inferiorly 1024, that is starting at a superior location 1014 or at a superior intercostal location 1016, and advancing the lead in an inferior direction. In either such example, the right ITV, left ITV, or both ITV vessels may be used, as indicated at 1026.

Other vessels and implanted lead locations may also be used (such as having a lead in the azygos vein, an intracardiac lead, a subcutaneous lead) or additional devices such as a separately implanted leadless cardiac pacemaker may be included as well. In a further example, one or more of the transverse veins that flow into the ITV may be used for placement of an electrode or lead. For example, upon accessing an ITV, a physician may further access and emplace a lead or electrode into one of the anterior intercostal veins which run along the intercostal spaces of the anterior chest.

In an example, attaching to an IPG may include attaching to a canister located in a subclavicular location 1032, historically a common place to put an implanted canister for a transvenous defibrillator or pacemaker. In another example, attaching to an IPG may include attaching to a canister located in an axillary position 1034, such as that used with the S-ICD System. Other IPG locations may be used. Attachment may be directly to the IPG or to a splitter, yoke, or lead extension, if desired.

In an example, test operation 1040 may be used to verify one or both of device functionality and efficacy. For example, sensing operations 1042 may be tested and configured to check for adequate signal availability, for example, or by setting gain, filtering, or sensing vector selection parameters. Defibrillation operations 1044 may be tested by inducting an arrhythmia such as a ventricular fibrillation to determine whether the device will sense the arrhythmia and, if the arrhythmia is sensed, to ensure that the device can adequately provide therapy output by delivering defibrillation at a preset energy. Defibrillation testing 1044 may include determining for a given patient an appropriate defibrillation threshold, and setting a parameter for therapy delivery at some safety margin above the defibrillation threshold.

Prior transvenous systems would typically deliver up to 35 Joules of energy at most, with storage of up to 40 Joules of energy, using peak voltages in the range of up to nearly 1000 volts. The S-ICD System can deliver up to 80 Joules of energy, with 65 Joules often used for in-clinic system testing, with a peak voltage in the range of 1500 volts. The ITV location may facilitate energy levels similar to those of traditional transvenous systems (5-35 Joules, approximately), or may be somewhat higher (5 to about 50 joules, for example), or may still be higher (10 to about 60 joules, for example). Pacing thresholds may also be closer to those for traditional transvenous systems than the more recent S-ICD System.

In an example, pacing testing operation 1046 may include determining which, if any, available pacing vectors are effective to provide pacing capture. If desired, parameters may be tested as well to determine and optimize settings for delivery of cardiac resynchronization therapy. This may include testing of pacing thresholds to optimize energy usage and delivery, as well as checking that adverse secondary effects, such as patient sensation of the delivered pacing or inadvertent stimulation of the phrenic nerve, diaphragm or skeletal muscles are avoided.

In some cases, the left and/or right ITV may be used to access the mediastinum. From such a position, beneath the rib cage, the amount of energy required for defibrillation and pacing efficacy would logically be lower than outside of the sternum and/or rib cage, since the mediastinum location is closer to the heart and bone is generally not a very good conductor of electrical energy, at least when speaking in terms of the tissues in the human body. Indeed, the insertion of a lead through the ITV (e.g., using any of superior access, inferior access, and/or intercostal access) may enable safe placement in the mediastinum.

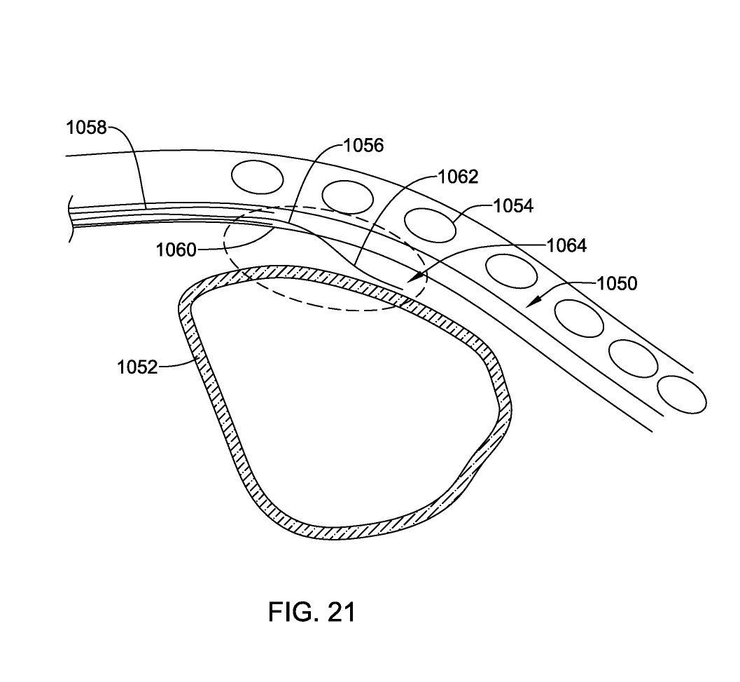

FIGS. 21-25 illustrate a method for placing a lead in the mediastinum. FIG. 21 is a lateral view of a portion of an illustrative method for placing a lead in the mediastinum through the left and/or right ITV. Referring now to FIG. 21, in this example, a patient is shown in a lateral view with relevant elements shown in isolation for clarity purposes. The ITV is shown at 1050 (item 1050 may be the left or right ITV), passing generally over the heart 1052 and beneath the ribs 1054. Access to the ITV 1050 may be achieved using any of the methods described above (e.g., superior access, inferior access, cut-down, intercostal access, etc.).

A guidewire 1056 is advanced through the ITV 1050 to a desired location adjacent to the heart 1052. The guidewire may be the same as used in gaining initial access to the vessel (if one is used to gain access), or may be a different guidewire. A guide catheter or sheath 1058 is advanced over the guidewire 1056. Once the guidewire 1056 is adjacent to the heart, the guidewire 1056 is advanced through the wall 1060 of the ITV 1050 such that the distal end region 1062 of the guidewire 1056 enters the mediastinal space 1064 between the pericardium (not explicitly shown) and the ITV 1050. In other words, the guidewire 1056 exits the ITV 1050. The target location in region 1064 generally contains some loose connective tissues (e.g., sternopericardiac ligaments), muscle, nerves and blood vessels. Anchoring a lead may be desirable, for example, in the region between the left and/or right ITV (and beneath the rib cage).

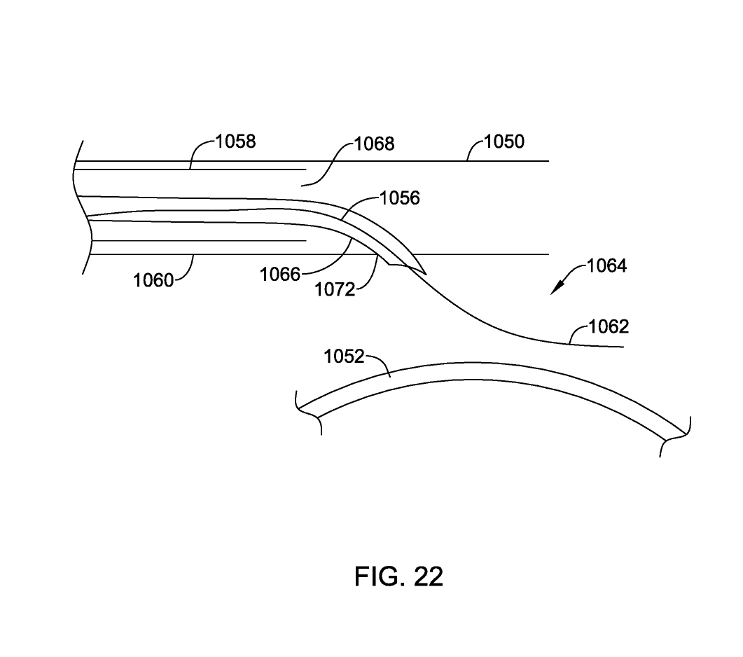

FIGS. 22-25 are enlarged views of the ITV 1050, heart 1052, and mediastinal space 1064 to provide greater detail on a delivery system approach. Referring now to FIG. 22, after the guidewire 1056 has been advanced into the mediastinal space 1064, a needle 1066, or other puncturing device, is advanced over the guidewire 1056, through the vessel wall 1060 and into the mediastinal space 1064 to create an opening or puncture 1072 in the vessel wall 1060. In some cases, the needle 1066 may be used to puncture the vessel wall 1060 prior to the guidewire 1056 exiting the ITV 1050. While the guidewire 1056 and/or needle 1066 are illustrated as exiting from a distal end opening 1068 of the guide catheter 1058, in some cases, the guidewire 1056 and/or needle 1066 may exit through a side port of the guide catheter 1058.

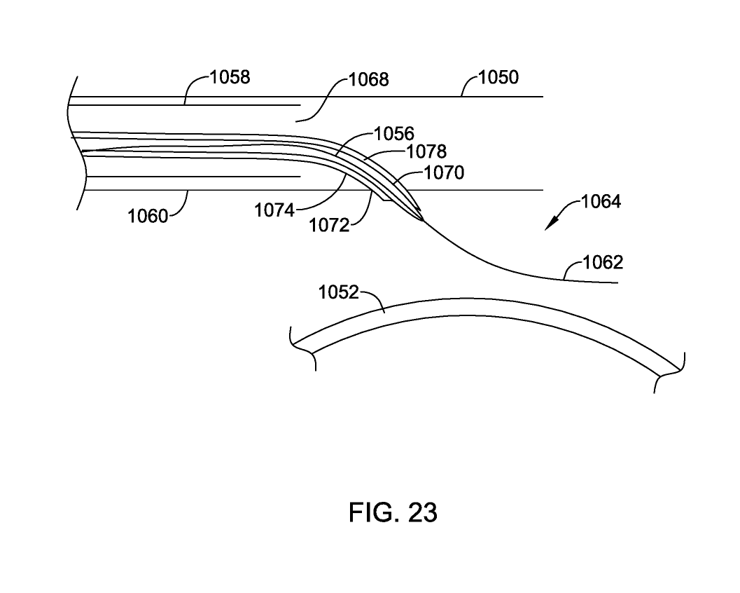

The needle 1066 may be retracted and an inner, or second, sheath 1074 and dilator 1070 advanced over the guidewire 1056, as shown in FIG. 23. In some cases, the inner sheath 1074 and the dilator 1070 may be advanced simultaneously. In other cases, the dilator 1070 may be advanced through the puncture 1072 prior to the inner sheath 1074, or vice versa.

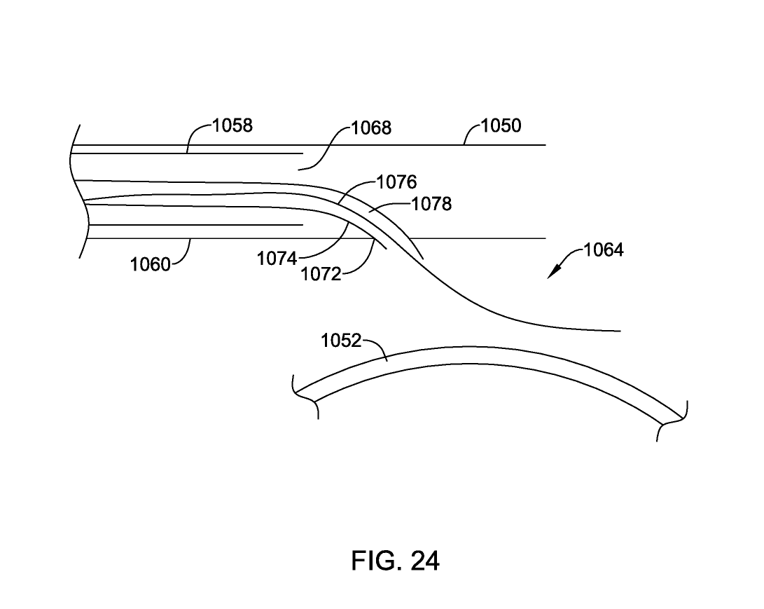

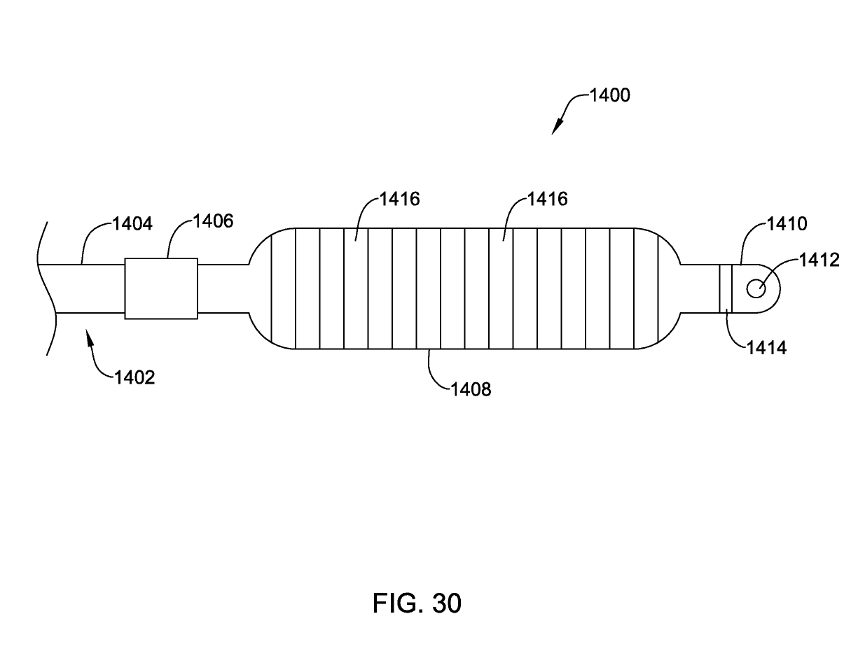

In some cases, it may be desirable to gently compress or push back the tissues 1063 to create an open space, recess, or cavity for receiving a larger lead and electrode assembly. The dilator 1070 and/or inner sheath 1074 may be used to displace the tissues (e.g., sternopericardiac ligaments) in the mediastinal space 1064. It is contemplated that the dilator 1070 and/or inner sheath 1074 may be or may function in a similar manner to a deflectable catheter. For example, the dilator 1070 and/or inner sheath 1074 may have a deflectable distal end region which allows the dilator 1070 and/or inner sheath 1074 to push through the tissues of the mediastinal space 1064. It is contemplated that in order to push through and/or displace the tissues of the mediastinal space 1064, the distal end region of the dilator 1070 and/or inner sheath 1074 may have a stiffness or rigidity that allows the distal end thereof to be pushed through the tissues. In some cases, the dilator 1070 and/or inner sheath 1074 may be rotated and/or deflected within the mediastinal space to further enlarge the open space, recess, or cavity created by the dilator 1070 and/or inner sheath 1074. It is contemplated that a wire, such a guidewire 1062, of sufficient stiffness may be used to create an open space, recess, or cavity within the mediastinal space 1064. A number of electrode structures having an increased surface area may be deployed in the space, recess, or cavity created by the dilator 1070, inner sheath 1074, and/or guidewire 1062. Various electrode structures are discussed in more detail with respect to FIGS. 30-36. The electrode structures may be implanted in a similar manner to that described with respect to FIGS. 24-29.

The guidewire 1056 and the dilator 1070 are removed from the guide catheter 1058 and a smaller diameter guidewire 1076 advanced through the inner sheath 1074, as shown in FIG. 24. In some instances, the first guidewire 1056 may have a diameter in the range of 0.030 to 0.040 inches (0.762 to 1.016 millimeters), or about 0.035 inches (0.889 millimeters) and the second guidewire 1076 may have a diameter in the range of 0.009 to 0.019 inches (0.229 to 0.483 millimeters), or about 0.014 inches (0.356 millimeters). These are just examples. The size of the guidewire used for each step may be dependent on the size of the device to be advanced over the guidewire 1056, 1076.

Various leads with a combination of electrodes and/or sensors may be delivered over the second guidewire 1076 and through the lumen 1078 of the second sheath 1074. The guidewire 1076 and the sheaths 1058, 1074 may be removed after placement of the lead. Blood loss through the puncture 1072 in the vessel wall 1060 may be of no consequence due to the low pressure in the ITV 1050. In other words, it may not be necessary to close or seal the puncture during device changes. For example, blood clotting may be sufficient to seal the puncture 1072. However, a suture sleeve may be used to close the puncture 1072.

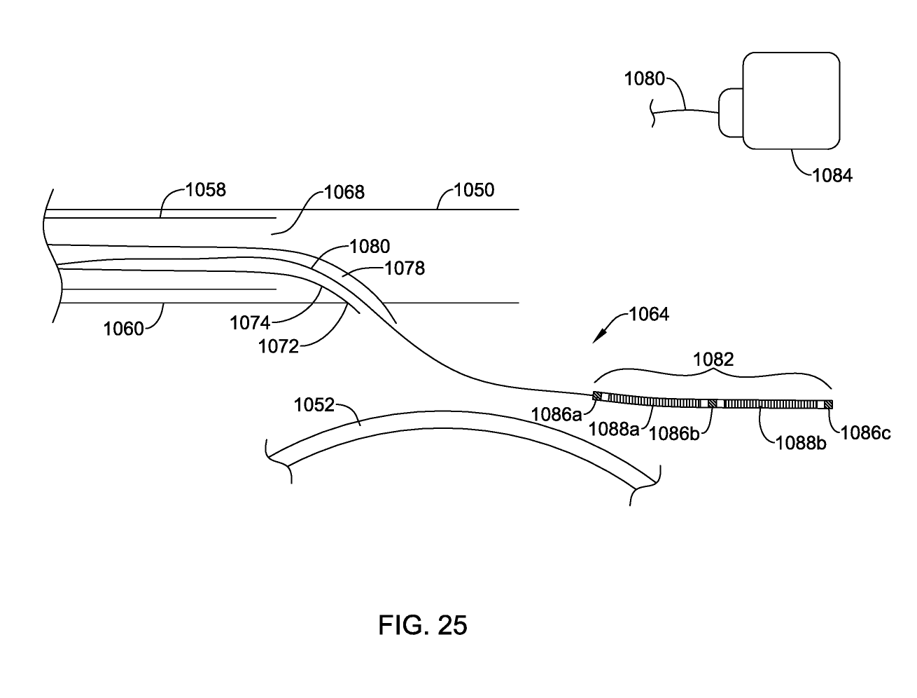

FIG. 25 shows implantation of an implantable cardiac stimulus device in the mediastinal space 1064, with the inner sheath 1074 and guide catheter 1058 still in place. The system includes an implantable pulse generator 1084 which may be placed in a subclavicular location, at the anterior axillary line, the midaxillary line, or in the posterior axillary line (or any other suitable position, as desired). The pulse generator 1084 may be placed as shown in U.S. patent application Ser. No. 15/667,221, titled PACEMAKERS FOR IMPLANT IN THE INTERNAL THORACIC VASCULATURE WITH COMMUNICATION TO OTHER IMPLANTABLE DEVICES, the disclosure of which is incorporated herein by reference.

A lead 1080 passes into the mediastinal space 1064 through the puncture 1072 in the vessel wall 1060. While the lead 1080 is described as being advanced over the guidewire 1076, the lead 1080 may be delivered to the mediastinal space 1064 with or without the use of a guidewire using any of the delivery mechanisms and methods described herein with respect to delivery in the ITV.

In the example, the lead 1080 includes a multi-electrode distal structure as shown at 1082. However, any of the lead designs described with respect to FIGS. 5 and 10-19 may be used. Further, while an anchoring mechanism is not explicitly shown, the lead can be fixated in the mediastinum using various means such as tines, hooks, biases, T-bar tethers, and other means. In addition to the engaging members described herein some illustrative additional anchoring mechanisms are discussed in US PG Patent Application Pub. No. 20170021159, titled SUBSTERNAL PLACEMENT OF A PACING AND/OR DEFIBRILLATING ELECTRODE, as well as US PG Patent Application Pub. No. 20170095657, titled FIXATION DEVICE FOR A SUBCUTANEOUS ELECTRODE, the disclosures of which are incorporated herein by reference.

In this example, the lead structure includes a proximal coil 1088A separate from a distal coil 1088B. The coils 1088A/B and canister 1084 may serve as therapy delivery electrodes. As such there may be multiple therapy vectors such as between coil 1088A and coil 1088B, between either of coils 1088A and 1088B and the canister 1084, or between a combination of two of the three therapy electrodes 1088A, 1088B and canister 1084, and the third such electrode, such as by linking coils 1088A and 1088B in common as the anode or cathode relative to the canister 1084.

A plurality of ring electrodes may be provided as shown at 1086A, 1086B, and 1086C. Electrode 1086C may also or instead be a tip electrode. Electrodes 1086A/B/C may serve as sensing electrodes. The coils 1088A, 1088B may also serve as sensing electrodes. These various electrodes may be used for sensing cardiac signals in various combinations using, for example, methods and circuitry discussed in U.S. Pat. No. 7,783,340, titled SYSTEMS AND METHODS FOR SENSING VECTOR SELECTION IN AN IMPLANTABLE MEDICAL DEVICE USING A POLYNOMIAL APPROACH, and U.S. Pat. No. 8,483,843, SENSING VECTOR SELECTION IN A CARDIAC STIMULUS DEVICE WITH POSTURAL ASSESSMENT, and/or US PG Patent Application Pub. Nos. 20170112399, 20170113040, 20170113050, and 20170113053, the disclosures of which are incorporated herein by reference.

In addition, one or more of the ring or tip electrodes 1086A, 1086B, 1086C may be used for therapy delivery. In an example, defibrillation therapy may use coils 1088A, 1088B coupled in common as the opposing pole to the canister 1084, while pacing therapy may use coils 1088A and 1086B as opposing electrodes for post-shock pacing therapy, with a still different combination of electrodes used to provide ventricular pacing therapy for example by pacing between coil 1088B and tip electrode 1086C. The lead 1080 may be placed as shown such that the proximal coil 1088A is about level with the atria, and distal coil 1088B is about level with the ventricles, if desired. In some examples fewer or different electrodes may be provided on the lead 1080 such as by excluding one or the other of the proximal coil 1088A or distal coil 1088B. Various designs are also shown herein. In some examples, one or more electrodes on the lead 1080 are provided at or inferior to the apex of the heart 1052, or at or superior to the top of the heart 1052.

In some cases, the lead 1080 may be placed on the left side of the patient. In other examples, the right side of the patient may instead or in addition be accessed, including the right ITV. Access to the right ITV may be achieved by advancing a guide catheter and/or guidewire from in any of the manners described herein.

In some examples, a lead 1080 may be placed adjacent to each of the left and right ITV and within the mediastinal space. In such an instance, a lead 1080 is delivered through each of the left and right ITV in a manner similar to that described with respect to FIGS. 21-25. Pacing between right and left side lead placements may be performed to target specific chambers or chamber combinations, or sensing may be performed using one pair of electrodes with therapy delivery using a different pair of electrodes to achieve resynchronization or other desirable effect.

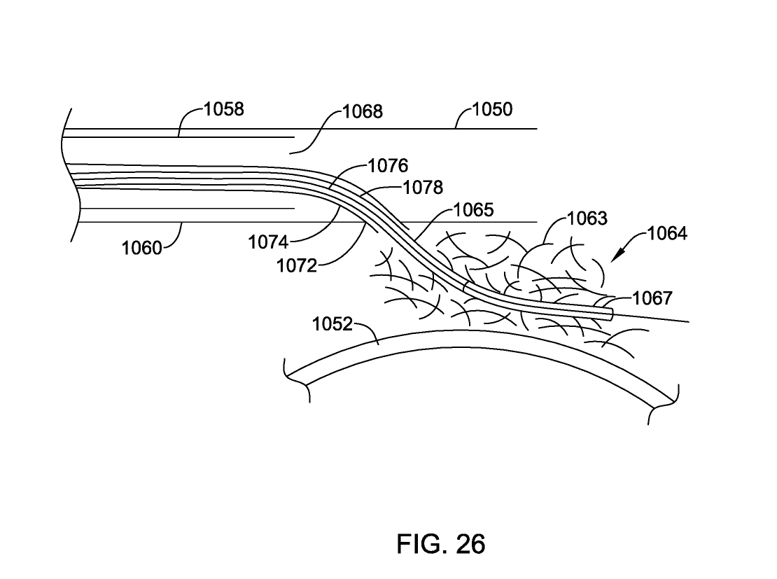

FIGS. 26-29 are enlarged views of the ITV 1050, heart 1052, and mediastinal space 1064 and a portion of the corresponding delivery system shown in the dashed lines in FIG. 21 illustrating another method for deploying a lead and/or electrode assembly in the mediastinum 1064. The target location (e.g., the mediastinum 1064) in region generally contains some loose connective tissues (e.g., sternopericardiac ligaments), muscle, nerves and blood vessels 1063. In some cases, it may be desirable to gently compress or push back the tissues 1063 to create an open space, recess, or cavity for receiving a larger lead and electrode assembly. The method for deploying the lead and/or electrode assembly may be the same as the method described with respect to FIGS. 21-24. However, once the second guidewire 1076 has been placed, an inflatable balloon catheter 1065 having an expandable balloon 1067 may be advanced over the guidewire 1076 (and within the lumen 1078 of the inner sheath 1074) and into the mediastinum 1064. The balloon catheter 1065 may take the shape of a traditional balloon catheter. For example, the balloon catheter 1065 may include an elongate shaft and an expandable member or balloon 1067 coupled to the shaft. When expansion is desired, the balloon 1067 may be filled with fluid and to remove the balloon 1067, the fluid may be removed to deflate the balloon 1067. It is contemplated that any arrangement of guidewire lumens, inflation lumens, recirculation lumens and/or deflation lumens may be used in the balloon catheter 1065.

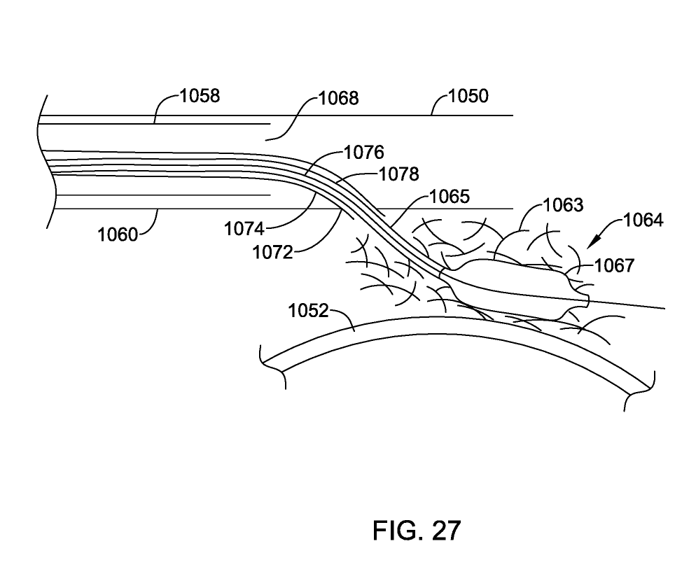

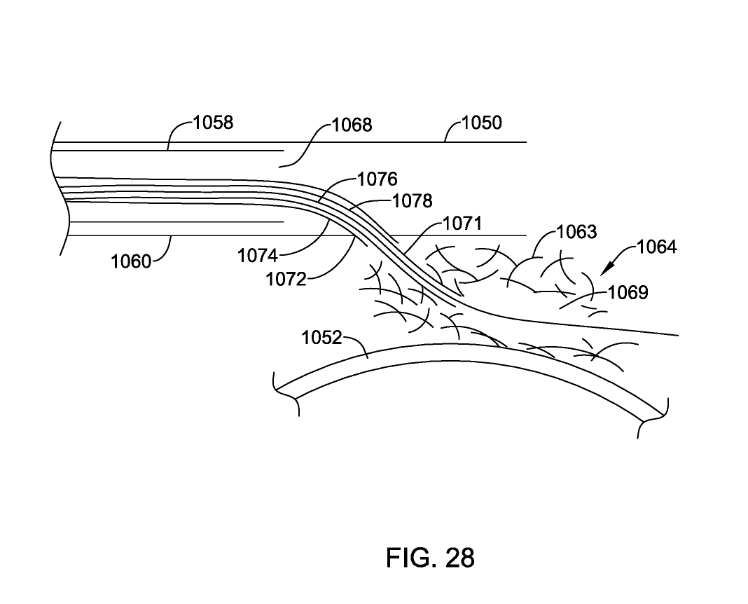

Once the expandable balloon 1067 is positioned adjacent to the target location (e.g., where the lead and/or electrode assembly is to be deployed) the balloon 1067 may be expanded, as shown in FIG. 27. Inflation of the balloon 1067 may push back the tissues 1063 (e.g., sternopericardiac ligaments). It is contemplated that the rate of inflation and/or pressure of the fluid inside the balloon 1067 may be adjusted to control the force applied to the tissues 1063. In other words, the balloon 1067 can be expanded such that the tissues 1063 are gently displaced and collateral damage to surrounding tissues is minimized. Once the tissue 1063 has been displaced, the balloon 1067 may be deflated and the balloon catheter 1065 removed from the inner sheath 1074 (and/or guide catheter 1058) leaving an open space 1069, as shown in FIG. 28, for receiving a lead and/or electrode assembly.

In some instances, another catheter or delivery device 1071 may be advanced through the inner sheath 1074 to facilitate delivery of the lead and/or electrode assembly. However, the use of an additional delivery device 1071 is not required. For example, the delivery device 1071 may be configured to maintain a self-expanding electrode structure in a collapsed configuration during delivery thereof. It is contemplated that a number of electrode structures having an increased surface area may be deployed in the space 1069. Various electrode structures are discussed in more detail with respect to FIGS. 30-36.

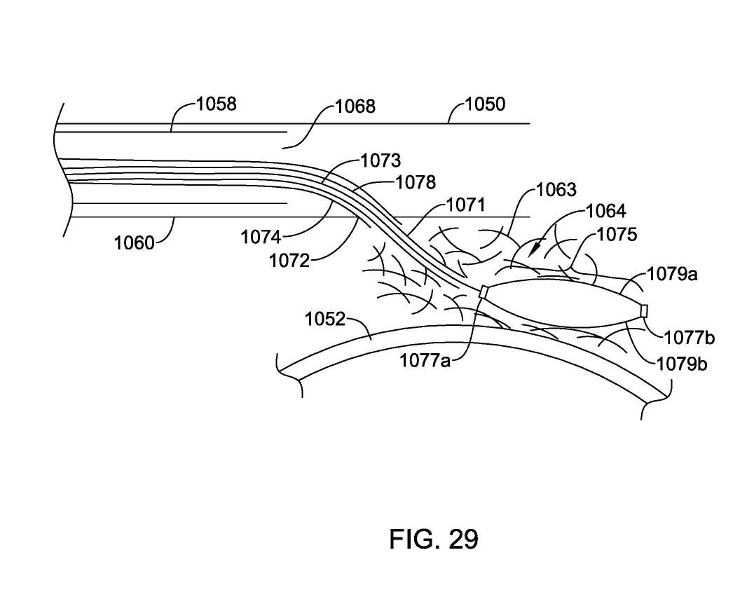

FIG. 29 shows implantation of an implantable cardiac stimulus device in the mediastinal space 1064, with the delivery device 1071, the inner sheath 1074, and guide catheter 1058 still in place. The system includes an implantable pulse generator (not explicitly shown) which may be placed in a subclavicular location, at the anterior axillary line, the midaxillary line, or in the posterior axillary line (or any other suitable position, as desired). A lead 1073 passes into the mediastinal space 1064 through the puncture 1072 in the vessel wall 1060. While the lead 1073 is described as being advanced over the guidewire 1076 in some cases, the lead 1073 may be delivered to the mediastinal space 1064 with or without the use of a guidewire using any of the delivery mechanisms and methods described herein with respect to delivery in the ITV.

In the example, the lead 1073 includes a multi-electrode distal structure as shown at 1075. However, any of the lead designs described with respect to FIGS. 5, 8B, 10-19, and 30-36 may be used. Further, while an anchoring mechanism is not explicitly shown, the lead can be fixated in the mediastinum using various means such as tines, hooks, biases, T-bar tethers, and other means. In addition to the engaging members described herein some illustrative additional anchoring mechanisms are discussed in US PG Patent Application Pub. No. 20170021159, titled SUB STERNAL PLACEMENT OF A PACING AND/OR DEFIBRILLATING ELECTRODE, as well as US PG Patent Application Pub. No. 20170095657, titled FIXATION DEVICE FOR A SUBCUTANEOUS ELECTRODE, the disclosures of which are incorporated herein by reference.

In this example, the lead structure includes a first coil 1079A separate from a second coil 1079B. The coils 1079A/B and canister may serve as therapy delivery electrodes. As such there may be multiple therapy vectors such as between coil 1079A and coil 1079B, between either of coils 1079A and 1079B and the canister, or between a combination of two of the three therapy electrodes 1079A, 1079B and canister, and the third such electrode, such as by linking coils 1079A and 1079B in common as the anode or cathode relative to the canister.

The electrode assembly 1075 may be configured to move between a collapsed or delivery configuration (not explicitly shown) and an expanded or implanted configuration, shown in FIG. 29. In some cases, the electrode assembly 1075 may be self-expanding. In other cases, the electrode assembly 1075 may be expanded through manipulation of an actuation mechanism.

A plurality of ring electrodes may be provided as shown at 1077A and Electrodes 1077A/B may serve as sensing electrodes. The coils 1079A, 1079B may also serve as sensing electrodes. These various electrodes may be used for sensing cardiac signals in various combinations using, for example, methods and circuitry discussed in U.S. Pat. No. 7,783,340, titled SYSTEMS AND METHODS FOR SENSING VECTOR SELECTION IN AN IMPLANTABLE MEDICAL DEVICE USING A POLYNOMIAL APPROACH, and U.S. Pat. No. 8,483,843, SENSING VECTOR SELECTION IN A CARDIAC STIMULUS DEVICE WITH POSTURAL ASSESSMENT, and/or US PG Patent Application Pub. Nos. 20170112399, 20170113040, 20170113050, and 20170113053, the disclosures of which are incorporated herein by reference.

In addition, one or more of the ring or tip electrodes 1077A, 1077B may be used for therapy delivery. In an example, defibrillation therapy may use coils 1079A, 1079B coupled in common as the opposing pole to the canister, while pacing therapy may use coils 1079A and 1077B as opposing electrodes for post-shock pacing therapy, with a still different combination of electrodes used to provide ventricular pacing.

In some cases, the lead 1073 may be placed on the left side of the patient. In other examples, the right side of the patient may instead or in addition be accessed, including the right ITV. Access to the right ITV may be achieved by advancing a guide catheter and/or guidewire from in any of the manners described herein.

In some examples, a lead 1073 may be placed adjacent to each of the left and right ITV and within the mediastinal space. In such an instance, a lead 1073 is delivered through each of the left and right ITV in a manner similar to that described with respect to FIGS. 26-29. Pacing between right and left side lead placements may be performed to target specific chambers or chamber combinations, or sensing may be performed using one pair of electrodes with therapy delivery using a different pair of electrodes to achieve resynchronization or other desirable effect.

FIGS. 30-36 illustrate various lead designs. These leads may be manufactured of any suitable material and by any suitable manner. For example, numerous polymers are known for lead manufacture. Internal longitudinal or lateral support members, such as braids, core wires, etc. may be provided. Extrusion or molding may be used. Internal conductors may be formed of any suitable material (stainless steel, titanium, gold, silver, or any other conductive material may be used) and may take any suitable form, such as simple wires, coated wires, braided or wound wires, drawn wires, and/or drawn filled tubes, or other structures. The leads may include on all or a portion thereof various coatings such as an anti-microbial coating to reduce the likelihood, severity, and/or progression of infection. Some illustrative lists for such design details follow later in the disclosure. In addition to the lead designs members described herein some illustrative additional lead designs are discussed in U.S. patent application Ser. No. 15/587,020, titled ELECTRODE DESIGNS IN IMPLANTABLE DEFIBRILLATOR SYSTEMS, the disclosure of which is incorporated herein by reference.

FIG. 30 shows a top view of an illustrative lead and electrode assembly 1400 for use with an implantable cardiac rhythm management system, such as, but not limited to the S-ICD System™ from Cameron Health, Inc., and Boston Scientific Corporation. The lead 1402 extends from a proximal end configured to connect to a canister through an intermediate region 1404 to a distal end having a proximal electrode 1406, a coil electrode 1408, and a distal tip electrode 1410. The positioning and/or spacing of the electrodes 1406, 1408, 1410 may be adjusted and/or reconfigured to optimize sensing and/or therapy delivery. For example, both sensing electrodes 1406, 1410 may be placed proximal to or distal to the coil electrode 1408. This is just an example. The distal tip electrode 1410 is shown with a suture hole 1412. The suture hole 1412 may be coupled to a base portion 1414. Other designs may be used. In some embodiments, a suture hole 1412, or other fixation means, may not be required and/or may not be provided.

As used herein, a coil electrode may be a helically wound element, filament, or strand. The filament forming the coil may have a generally round or a generally flat (e.g. rectangular) cross-sectional shape, as desired. However, other cross-sectional shapes may be used. The coil electrode may have a closed pitch, or in other words, adjacent windings may contact one another. Alternatively, the coil electrode may have an open pitch such that adjacent windings are spaced a distance from one another. The pitch may be uniform or varied along a length of the coil electrode. A varied pitch may include gradual tapered changes in pitch or abrupt or step-wise changes in pitch.

The shocking coil electrode 1408 may have a generally flattened cross-sectional configuration, although this is not required. The coil electrode 1408 may be formed from a round or flat (ribbon) wire, as desired. In some embodiments, the coil electrode 1408 may be formed as a subassembly and placed over the lead body 1402. Alternatively, the coil electrode 1408 may be formed as a unitary structure with or otherwise formed over the lead body 1402. While not explicitly shown, the coil electrode 1408 may include a lumen or passageway for receiving a stylet or other delivery aid. In some instances, adjacent windings 1416 of the coil electrode 1408 may be in contact with one another while in other instances adjacent windings 1416 may be spread out or spaced a distance from one another, as desired.

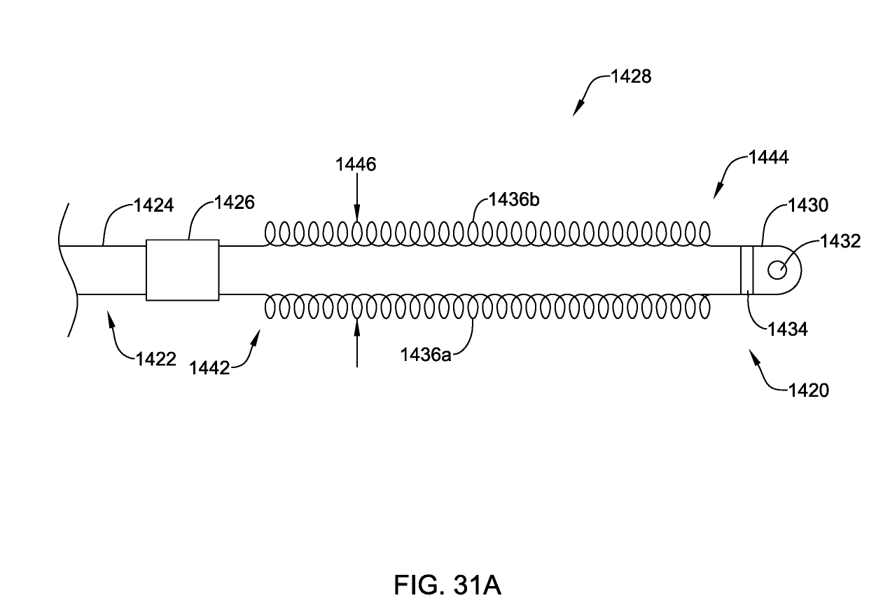

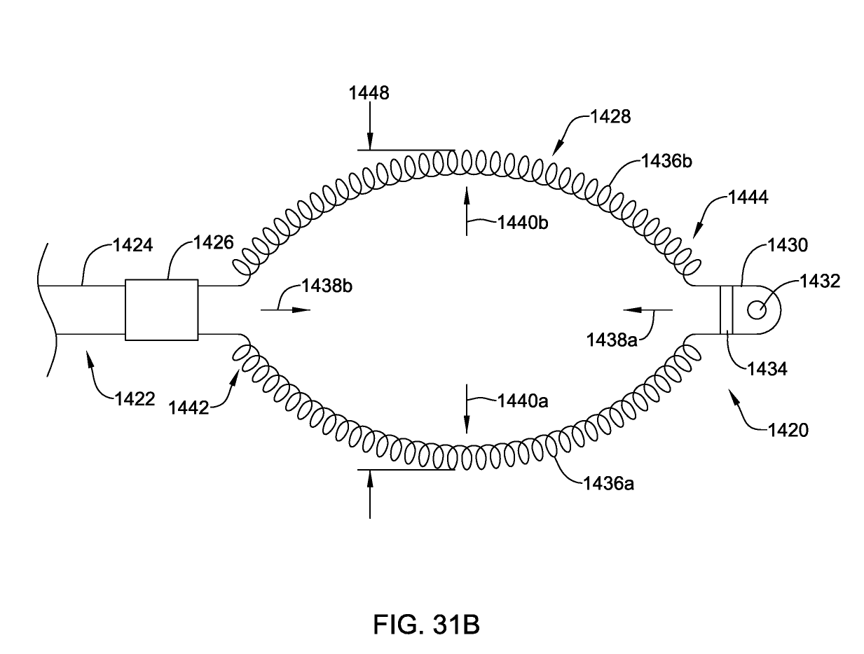

FIGS. 31A and 31B show a top view of another illustrative lead and electrode assembly 1420 for use with an implantable cardiac rhythm management system. In some embodiments, the illustrated assembly 1420 may be configured to move between a collapsed or delivery configuration, shown in FIG. 31A and an expanded or implanted configuration, shown in FIG. 31B. However, it is contemplated that the illustrative lead and electrode assembly 1420 of FIG. 31A may be both the delivery configuration and the implanted configuration. Similarly, the illustrative lead and electrode assembly 1420 of FIG. 31B may be both the delivery configuration and the implanted configuration.

The lead 1422 extends from this proximal end configured to connect to a canister through an intermediate region 1424 to a distal end having a proximal electrode 1426, a coil electrode 1428, and a distal tip electrode 1430. The positioning and/or spacing of the electrodes 1426, 1428, 1430 may be adjusted and/or reconfigured to optimize sensing and/or therapy delivery. For example, both sensing electrodes 1426, 1430 may be placed proximal to or distal to the coil electrode 1428. This is just an example. It is contemplated that the electrodes 1426, 1428, 1430 may be placed beneath the skin and over the ribcage of the patient. In other embodiments, the electrodes 1426, 1428, 1430 may be placed in a substernal location using an implant procedure that may include a xiphoid or sub-xiphoid incision that allows for tunneling along the back side of the sternum. The electrodes 1426, 1428, 1430 may also be placed elsewhere as desired including for example, for use with right sided, anterior-posterior, or other implant positions.

The distal tip electrode 1430 is shown with a suture hole 1432. The suture hole 1432 may be coupled to a base portion 414. Other designs may be used. In some embodiments, a suture hole 1432, or other fixation means, may not be required and/or may not be provided.

The coil electrode 1428 may be formed from two or more individual coil electrodes 1436a, 1436b. While the coil electrode 1428 is illustrated as including two coil electrodes 1436a, 1436b, the coil electrode 1428 may including any number of individual coil electrodes desired, such as, but not limited to, one, two, three, four, five, or more. Further, in either configuration, the coil electrodes 1436a, 1436b may be positioned close to one another (e.g. touching) or spaced a distance, as desired. The coil electrode 1428 may be affixed to the lead body 1422 at its proximal end 1442 and its distal end 1444. While not explicitly shown, in some embodiments, the lead body 1422 may include a portion that extends between the proximal end 1442 and the distal end 1444 of the coil electrode 1428. It is contemplated that the lead body 1422 may include a telescoping feature or nested tubular members that allows the proximal end 1442 and/or distal end 1444 of the coil electrode 1428 to be moved along a longitudinal axis of the system 1420, such as in the direction of arrows 1438a, 1438b, shown in FIG. 31B. In other embodiments, the lead body 1422 may be disposed within one or both of the coil electrodes 1436a, 1436b. While not explicitly shown, the coil electrode 1428 may include a lumen or passageway for receiving a stylet or other delivery aid.

The coil electrodes 1436a, 1436b may be actuatable or expandable from a delivery configuration having a first width 1446, shown in FIG. 31A, to an implanted configuration having a second larger width 1448, as shown in FIG. 31B. While the embodiments shown in FIGS. 31A and 31B are described as movable between two different configurations, it is contemplated the lead and electrode assembly 1420 may be fixed in either arrangement. In other words, in some embodiments the electrodes 1436a, 1436b may be movable relative to one another while in other embodiments, the electrodes 1436a, 1436b may be in a fixed arrangement relative to one another. It is contemplated that the coil electrode 1428, in either the delivery configuration or the implanted configuration, may be similar in size to the coil electrode 308 described above. The coil electrode 1428 may have a larger surface area and/or shadow than a typical shocking coil electrode. It is contemplated that increasing the surface area and/or shadow may allow the defibrillation threshold to be lowered which may allow the canister, such as canister 12, to have a smaller profile.

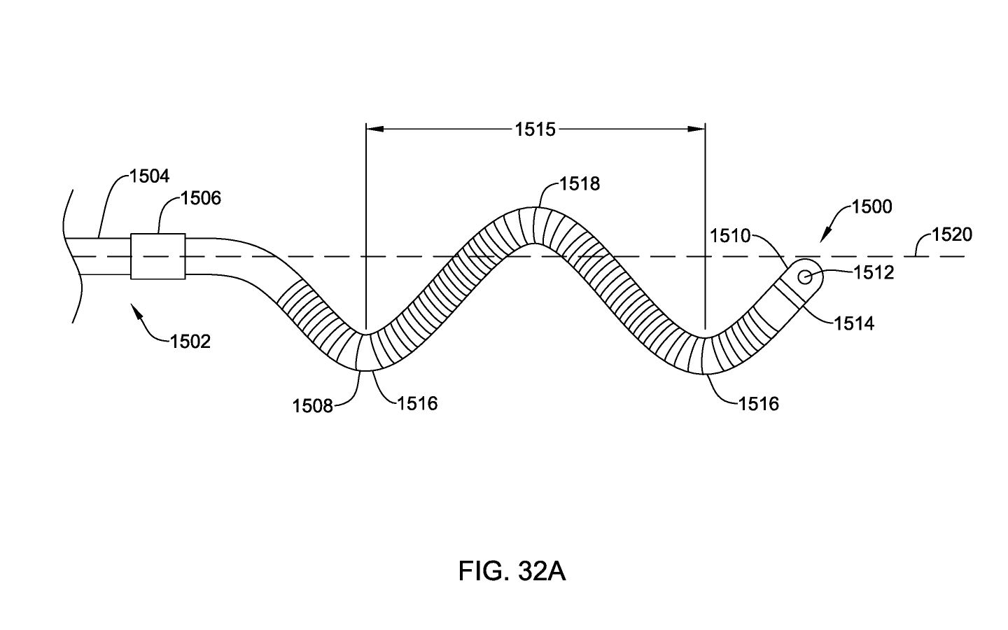

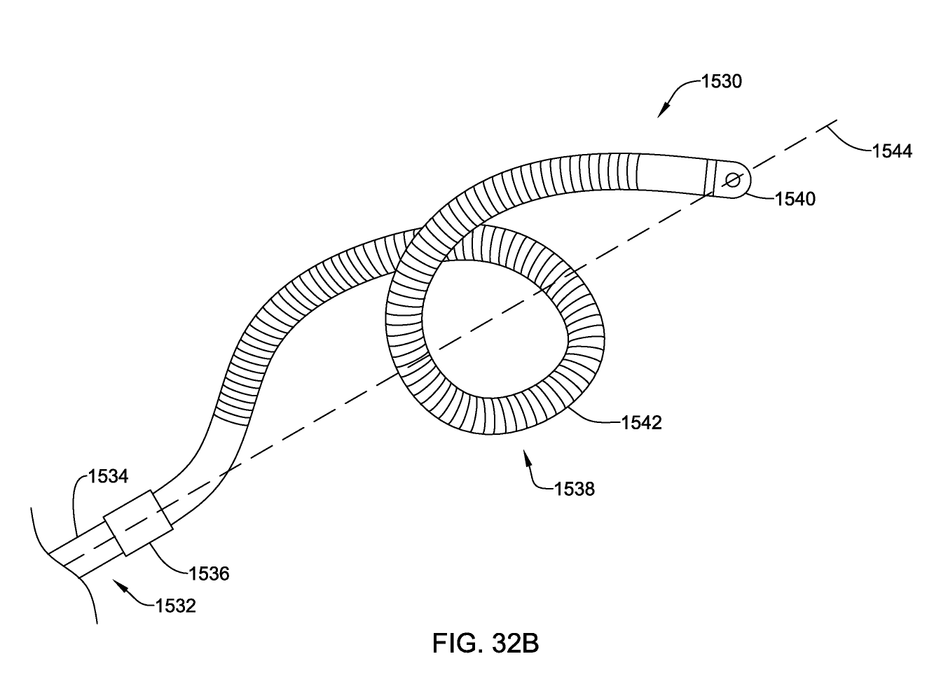

FIG. 32A shows a top view of another illustrative lead and electrode assembly 1500 for use with an implantable cardiac rhythm management system. While not explicitly shown, the illustrated assembly 1500 may be configured to move between a delivery configuration and an implanted configuration. For example, the illustrated assembly 1500 may be delivered in a generally linear configuration and placed into the oscillating configuration shown in FIG. 32A after deployment. This may allow a smaller delivery tool to be used for insertion of the lead assembly 1500. However, this is not required. It is contemplated that the illustrative lead and electrode assembly 1500 may be delivered in the oscillating or curved configuration.

The lead 1502 extends from a proximal end configured to engage a canister through an intermediate region 1504 to a distal end having a proximal electrode 1506, a coil electrode 1508, and a distal tip electrode 1510. The positioning and/or spacing of the electrodes 1506, 1508, 1510 may be adjusted and/or reconfigured to optimize sensing and/or therapy delivery. For example, both sensing electrodes 1506, 1510 may be placed proximal or distal to the coil electrode 1508. This is just an example.

The distal tip electrode 1510 is shown with a suture hole 1512. The suture hole 1512 may be coupled to a base portion 1514. Other designs may be used. In some embodiments, a suture hole 1512, or other fixation means, may not be required and/or may not be provided.

The coil electrode 1508 have a generally oscillating shape. For example, the coil electrode 1508 may include one or more oscillations 1515 each having a peak 1516 and a valley 1518. The oscillations 1515 may be uniformly positioned along the longitudinal to axis 1520 of the assembly 1500 along a least a portion of the length of the coil electrode 1508. In such an instance, the peak 1516 and valley 1518 may have the same “height” or peak amplitude (as measured from the longitudinal axis 1520). Alternatively, or additionally, the oscillations may be shifted from the longitudinal axis 1520 such that either the peak 1516 or the valley 1518 has a greater peak amplitude than the other along a least a portion of the length of the coil electrode 1508. The frequency of the oscillations 1515 may also be varied. For example, the frequency of the oscillations 1515 may be increased such that there are more oscillations over a similar length. It is contemplated that the coil electrode 1508 may include less than one, one, two, three, four, five, or more oscillations, as desired. It is further contemplated that the frequency of the oscillations 1515 may be varied along the length of a coil electrode 1508. Any combination of frequency, peak amplitude, and/or offsets from the longitudinal axis 1520 may be used to arrive at the desired shape.

The coil electrode 1508 may have a larger surface area and/or shadow than a typical shocking coil electrode. It is contemplated that increasing the surface area and/or shadow may allow the defibrillation threshold to be lowered which may allow the canister to have a smaller profile.

In some embodiments, the coil electrode 1508 may be delivered in a straightened, or generally linear, configuration. This may allow the assembly 1500 to be implanted using a smaller profile delivery device. In one example, the distal electrode 1510 may be secured to the tissue and subsequently the lead body 1502 may be distally advanced to apply a pushing force to the proximal end region of the coil electrode 1508. This may cause the coil electrode 1508 to wind back and forth, as shown in FIG. 32A, while also shortening in length. It is contemplated that the same result may be achieved by fixing the proximal end and applying a proximal, or pulling force to the distal end of the coil electrode 1508. In yet another example, the coil electrode 1508 may be formed in the oscillating configuration illustrated in FIG. 32A. The coil electrode 1508 may be compressed into a lower profile delivery configuration through the application of a biasing force. For example, when the coil electrode 1508 are disposed within a delivery tool, the delivery tool may maintain the coil electrode 1508 in a reduced profile configuration (e.g. elongated or compressed). In yet another embodiment, the coil electrode 1508 may be implanted in its oscillating configuration using a delivery tool wide enough to house the coil electrode 1508 in its oscillating configuration.

FIG. 32B shows a top view of another illustrative lead and electrode assembly 1530 for use with an implantable cardiac rhythm management system. While not explicitly shown, the illustrated assembly 1530 may be configured to move between a delivery configuration and an implanted configuration. For example, the illustrated assembly 1530 may be delivered in a generally linear configuration and placed into the helical configuration shown in FIG. 32B after deployment. This may allow a smaller delivery tool to be used for insertion of the lead assembly 1530. However, this is not required. It is contemplated that the illustrative lead and electrode assembly 1530 may be delivered in the helical configuration.

The lead 1532 extends from a proximal end through an intermediate region 1534 to a distal end having a proximal electrode 1536, a coil electrode 1538, and a distal tip electrode 1540. The positioning and/or spacing of the electrodes 1536, 1538, 1540 may be adjusted and/or reconfigured to optimize sensing and/or therapy delivery. For example, both sensing electrodes 1536, 1540 may be placed proximal or distal to the coil electrode 1538. This is just an example. It is contemplated that the electrodes 1536, 1538, 1540 may be placed beneath the skin and over the ribcage of the patient. In other embodiments, the electrodes 1536, 1538, 1540 may be placed in a substernal location using an implant procedure that may include a xiphoid or sub-xiphoid incision that allows for tunneling along the back side of the sternum. The electrodes 1536, 1538, 1540 may also be placed elsewhere as desired including for example, for use with right sided, anterior-posterior, or other implant positions.

The coil electrode 1538 have a generally helical shape. For example, the coil electrode 1538 may be wound into a helix 1542. The helix 1524 may have a three dimensional shape which may facilitate better contact with the facial plane. The coil electrode 1538 forming the helix 1542 may have a generally round or a generally flat (e.g. rectangular) cross-sectional shape, as desired. However, other cross-sectional shapes may be used. The helix 1542 may have a closed pitch, or in other words, adjacent windings may contact one another. Alternatively, the helix 1542 may have an open pitch such that adjacent windings are spaced a distance from one another. The pitch may be uniform or varied along a length of the coil electrode. A varied pitch may be gradual tapered changes in pitch or abrupt or step-wise changes in pitch. The helix 1542 may include any number of windings desired, such as, but not limited to less than one, one, two, three, four, or more.

The windings of the helix 1542 may be uniformly positioned (e.g. centered) along the longitudinal axis 1544 of the assembly 1530 along a least a portion of the length of the coil electrode 1538. Alternatively, or additionally, the helix 1542 may be shifted from the longitudinal axis 1544 such the center of the helix 1542 is offset from the longitudinal axis 1544 along a least a portion of the length of the coil electrode 1538. Any combination of pitch, winding diameter, and/or offsets from the longitudinal axis 1544 may be used to arrive at the desired shape.

The coil electrode 1538 may have a larger surface area and/or shadow than a typical shocking coil electrode. It is contemplated that increasing the surface area and/or shadow may allow the defibrillation threshold to be lowered which may allow the canister to have a smaller profile.

In some embodiments, the coil electrode 1538 may be delivered in a straightened, or generally linear, configuration. This may allow the assembly 1530 to be implanted using a smaller profile delivery device. In one example, the distal electrode 1540 may be secured to the tissue and subsequently the lead body 1532 may be distally advanced to apply a pushing force to the proximal end region of the coil electrode 1538. This may cause the coil electrode 1538 to coil, as shown in FIG. 32B while also shortening in length. It is contemplated that the same result may be achieved by fixing the proximal end and applying a proximal, or pulling force to the distal end of the coil electrode 1538. In yet another example, the coil electrode 1538 may be formed in the helical configuration illustrated in FIG. 32B. The coil electrode 1538 may be compressed (e.g. elongated or stretched) into a lower profile delivery configuration through the application of a biasing force. For example, when the coil electrode 1538 are disposed within a delivery tool, the delivery tool may maintain the coil electrode 1538 in a reduced profile configuration (e.g. elongated, compressed, stretched, etc.). In yet another embodiment, the coil electrode 1538 may be implanted in its helical configuration using a delivery tool wide enough to house the coil electrode 1538 in its helical configuration.

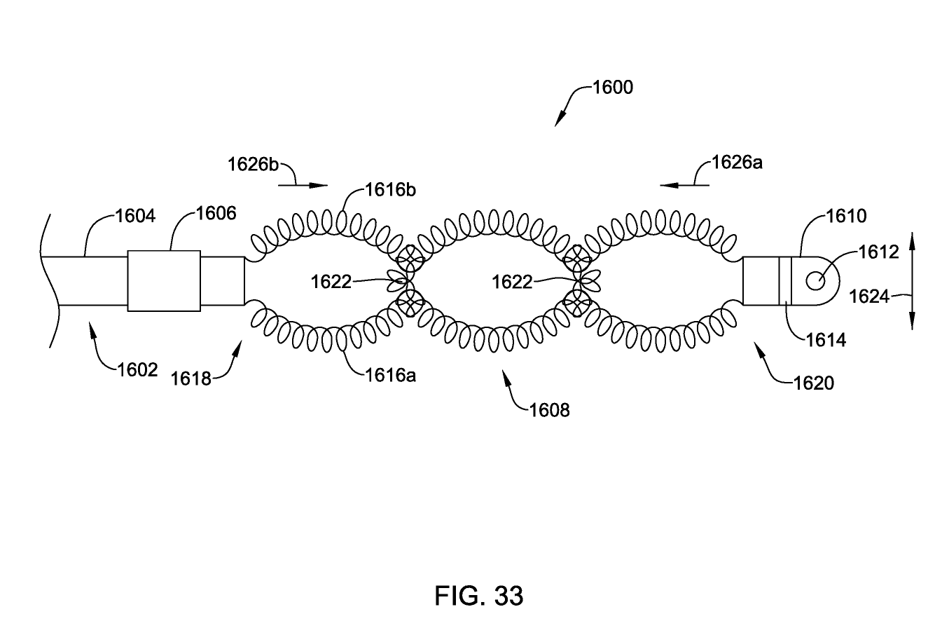

FIG. 33 shows a top view of another illustrative lead and electrode assembly 1600 for use with an implantable cardiac rhythm management system. In some embodiments, the illustrated assembly 1600 may be configured to move between a collapsed or delivery configuration and an expanded or implanted configuration. FIG. 33 illustrates a configuration between the collapsed and fully expanded configuration.

The lead 1602 extends from a proximal end configured to engage a canister through an intermediate region 1604 to a distal end having a proximal electrode 1606, a coil electrode 1608, and a distal tip electrode 1610. The positioning and/or spacing of the electrodes 1606, 1608, 1610 may be adjusted and/or reconfigured to optimize sensing and/or therapy delivery. For example, both sensing electrodes 1606, 1610 may be placed proximal to or distal to the coil electrode 1608. This is just an example.

The distal tip electrode 1610 is shown with a suture hole 1612. The suture hole 1612 may be coupled to a base portion 1614. Other designs may be used. In some embodiments, a suture hole 1612, or other fixation means, may not be required and/or may not be provided.

The coil electrode 1608 may be formed from two or more individual coil electrodes 1616a, 1616b. While the coil electrode 1608 is illustrated as including two coil electrodes 1616a, 1616b, the coil electrode 1608 may including any number of individual coil electrodes desired, such as, but not limited to, one, two, three, four, five, or more. The coil electrodes 1616a, 1616b may have a generally two dimensional oscillatory configuration, similar in form and function to the oscillatory configuration described with respect to FIG. 32A. Alternatively, the coil electrodes 1616a, 1616b may have a generally three dimensional helical configuration, similar in form and function to the helical configuration described with respect to FIG. 32B. The coil electrodes 616a, 616b may be wound or coiled in opposite directions such that the coil electrodes 616a, 616b cross at cross points 1622. In some embodiments, the coil electrodes 616a, 616b may be secured to one another at the cross points 1622, although this is not required. It is contemplated that the coil electrode 1608 may include any number of cross points 1622 desired, such as, but not limited to one, two, three, four, or more.

The coil electrode 1608 may be affixed to the lead body 1602 at its proximal end 1618 and its distal end 1620. While not explicitly shown, in some embodiments, the lead body 1602 may include a portion that extends between the proximal end 1618 and the distal end 1620 of the coil electrode 1608. It is contemplated that the lead body 1602 may include a telescoping feature or nested tubular members that allows the proximal end 1618 and/or distal end 1620 of the coil electrode 608 to be moved along a longitudinal axis of the system 1600, such as in the direction of arrows 1626a, 1626b, shown in FIG. 33. In other embodiments, the lead body 1602 may be disposed within one or both of the coil electrodes 1616a, 1616b. While not explicitly shown, the coil electrode 1608 may include a lumen or passageway for receiving a stylet or other delivery aid.

The coil electrodes 1616a, 1616b may be actuatable or expandable from a delivery configuration having a first width to an implanted configuration having a second larger width in the direction of arrow 1624. The coil electrode 1608 may have a larger surface area and/or shadow than a typical shocking coil electrode. It is contemplated that increasing the surface area and/or shadow may allow the defibrillation threshold to be lowered which may allow the canister to have a smaller profile.

The lead and electrode assembly 1600 may be actuated between the delivery configuration and the implanted configuration using any number of deployment mechanisms. In one example, the distal electrode 1610 may be secured to the tissue. Once the distal end has been secured, the lead body 1602 may be distally advanced to apply a pushing force to the proximal end 1618 of the coil electrode 1608. This may cause the coil electrodes 1616a, 1616b to bias outward, as shown at arrow 1624 while also shortening in length, as shown at arrows 1626a, 1626b. It is contemplated that the same result may be achieved by applying a proximal, or pulling force to the distal end 1620 of the coil. In yet another example, the coil electrodes 1616a, 1616b may be formed in the expanded configuration. The coil electrodes 1616a, 1616b may be compressed into a lower profile delivery configuration through the application of a biasing force. For example, when the coil electrodes 1616a, 1616b are disposed within a delivery tool, the delivery tool may maintain the coil electrodes 1616a, 1616b in a reduced profile configuration.

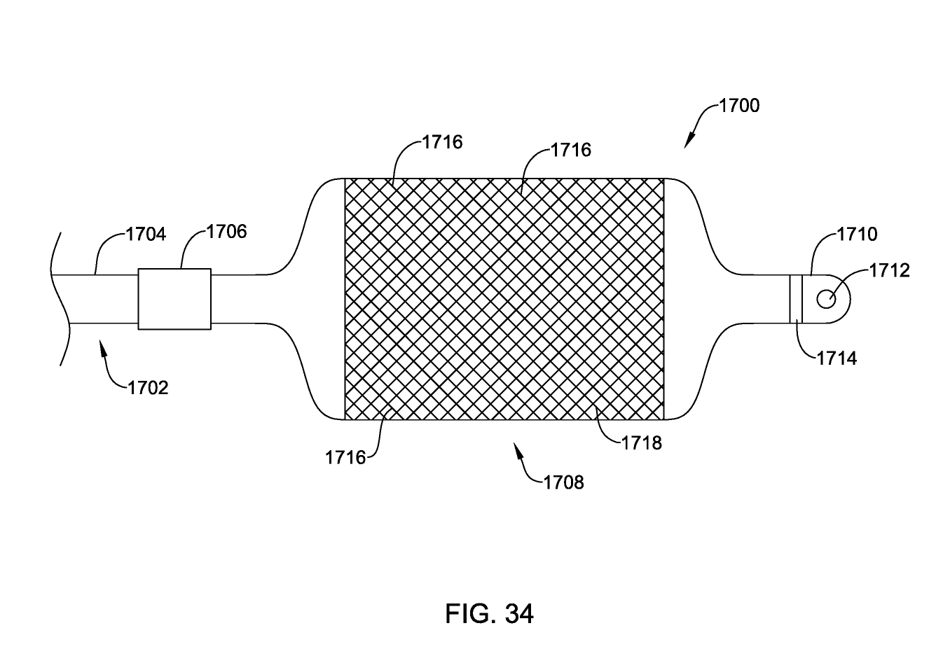

FIG. 34 shows a top view of another illustrative lead and electrode assembly 1700 for use with an implantable cardiac rhythm management system. While not explicitly shown, the illustrated assembly 1700 may be configured to move between a delivery configuration and an implanted configuration. For example, the illustrated assembly 1700 may be delivered in a generally collapsed configuration (e.g., rolled) and placed into the configuration shown in FIG. 34 after deployment. This may allow a smaller delivery tool to be used for insertion of the lead assembly 1700. However, this is not required. It is contemplated that the illustrative lead and electrode assembly 1700 may be delivered through a wide tunnel delivery tool with the shocking electrode 1708 in a carrier.

The lead 1702 extends from a proximal end through an intermediate region 1704 to a distal end having a proximal electrode 1706, a shocking electrode 1708, and a distal tip electrode 1710. The positioning and/or spacing of the electrodes 1706, 1708, 1710 may be adjusted and/or reconfigured to optimize sensing and/or therapy delivery. For example, both sensing electrodes 1706, 1710 may be placed proximal or distal to the shocking electrode 1708. This is just an example.

The distal tip electrode 1710 is shown with a suture hole 1712. The suture hole 1712 may be coupled to a base portion 1714. Other designs may be used. In some embodiments, a suture hole 1712, or other fixation means, may not be required and/or may not be provided.

The shocking electrode 1708 have a generally woven structure. For example, the shocking electrode 1708 may have a woven structure, fabricated from one or more filaments 1716. The filaments 1716 may be embedded in, or partially embedded in a silicone carrier 1718, although this is not required. In some embodiments, the shocking electrode 1708 may be braided with one filament 1716. In other embodiments, the shocking electrode 1708 may be braided with several filaments 1716. In another embodiment, the shocking electrode 1708 may be knitted or of a knotted type. The filaments 1716 may be have a generally round or a generally flat (e.g. rectangular) cross-sectional shape, as desired. However, other cross-sectional shapes may be used. In some embodiments, each filament 1716 may include a plurality of filaments wound or woven together. In still another embodiment, the shocking electrode 1708 may be laser cut. It is contemplated that a custom laser cut plate may be used to achieve desired mechanical properties as well as to arrive at shape which reduces the defibrillation threshold. While the shocking electrode 1708 is illustrated as having a substantially rectangular peripheral shape, the shocking electrode 1708 may take any shape desired such as, but not limited to ovular, circular, square, polygonal, etc. The shocking electrode 1708 may have a larger surface area and/or shadow than a typical shocking coil electrode. It is contemplated that increasing the surface area and/or shadow may allow the defibrillation threshold to be lowered which may allow the canister to have a smaller profile.

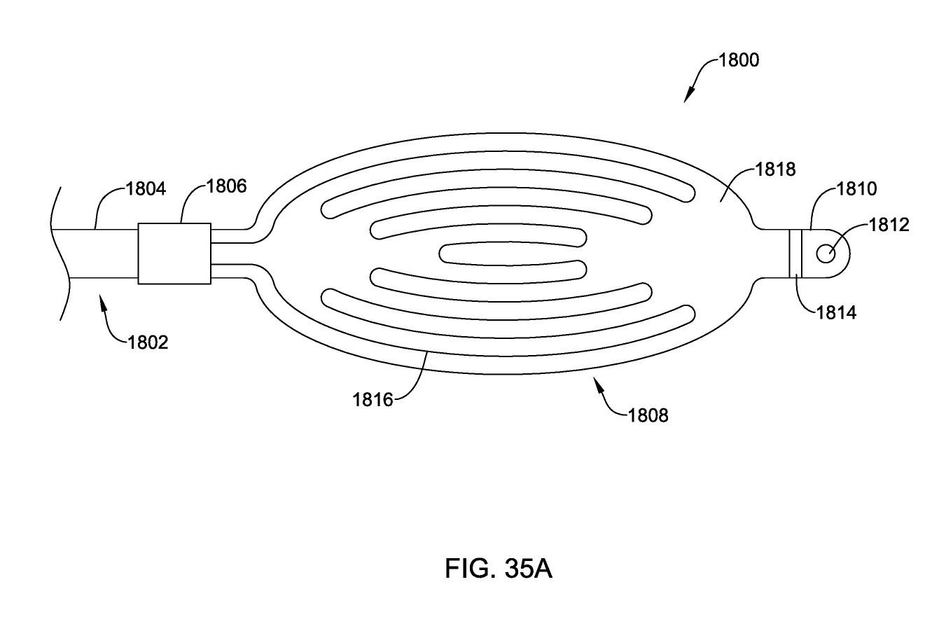

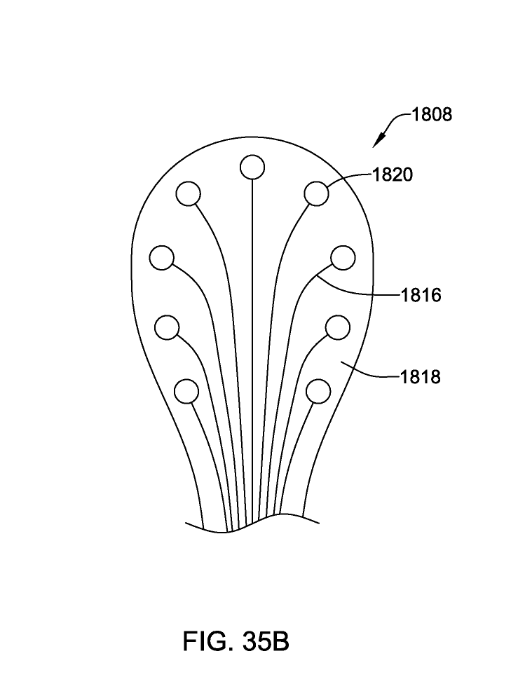

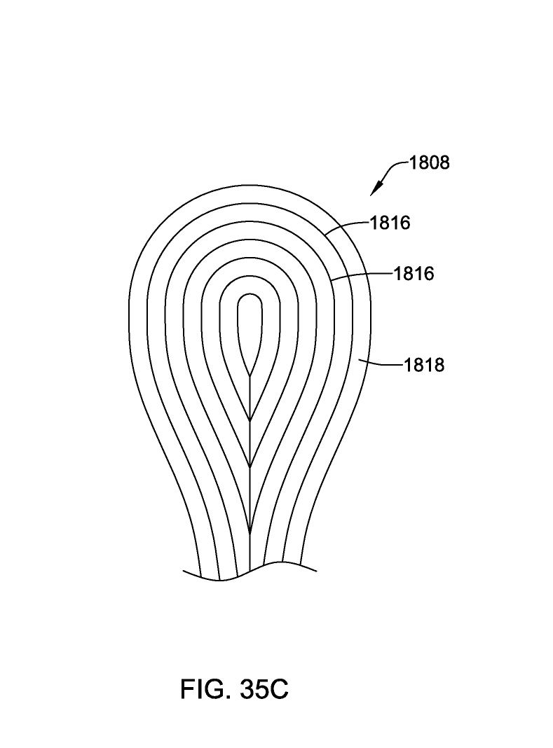

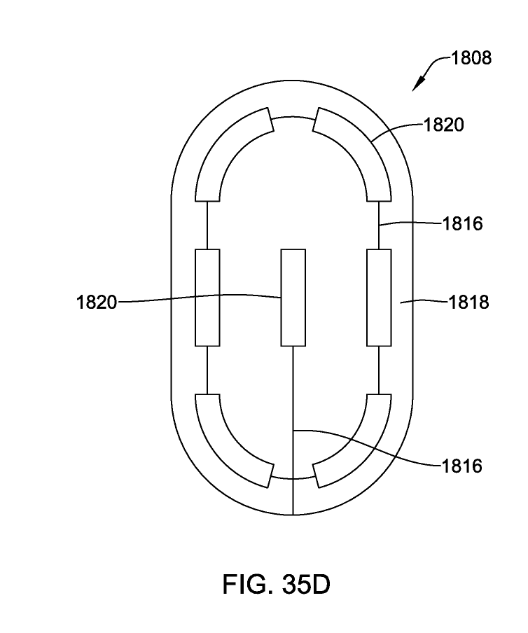

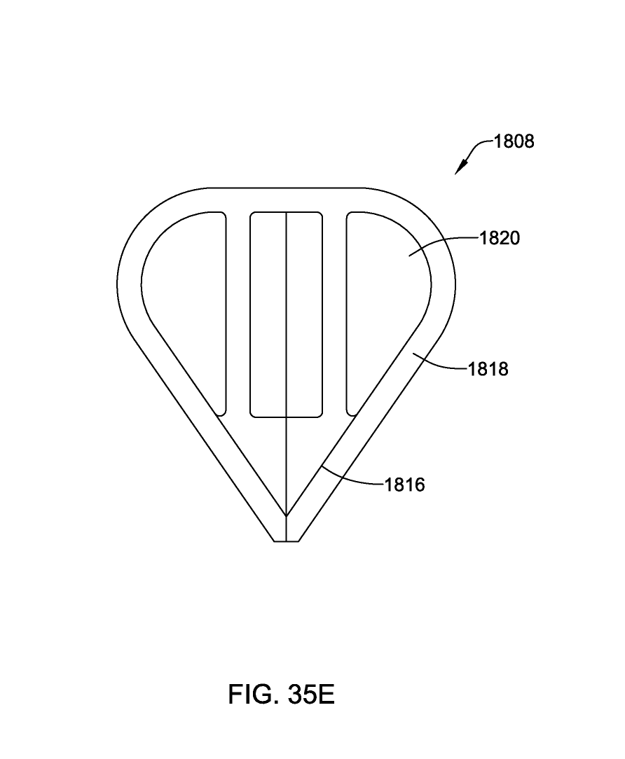

FIG. 35A shows a top view of another illustrative lead and electrode assembly 1800 for use with an implantable cardiac rhythm management system, such as, but not limited to the S-ICD System™ from Cameron Health, Inc., and Boston Scientific Corporation described with respect to FIG. 1. While not explicitly shown, the illustrated assembly 1800 may be configured to move between a delivery configuration and an implanted configuration. For example, the illustrated assembly 1800 may be delivered in a generally collapsed configuration (e.g. rolled) and placed into the configuration shown in FIG. 35A after deployment. This may allow a smaller delivery tool to be used for insertion of the lead assembly 1800. However, this is not required. It is contemplated that the illustrative lead and electrode assembly 1800 may be delivered through a wide tunnel delivery tool with the shocking electrode 1808 in a carrier.

The lead 1802 extends from a proximal configuration through an intermediate region 1804 to a distal end having a proximal electrode 1806, a shocking electrode 1808, and a distal tip electrode 1810. The positioning and/or spacing of the electrodes 1806, 1808, 1810 may be adjusted and/or reconfigured to optimize sensing and/or therapy delivery. For example, both sensing electrodes 1806, 1810 may be placed proximal to or distal to the shocking electrode 1808. This is just an example.

The distal tip electrode 1810 is shown with a suture hole 1812. The suture hole 1812 may be coupled to a base portion 1814. Other designs may be used. In some embodiments, a suture hole 1812, or other fixation means, may not be required and/or may not be provided.

The shocking electrode 1808 may be a printed circuit patch on a liquid crystal polymer 1818. The shocking electrode 1808 may include a platinum, gold, or other noble trace 1816 positioned on the liquid crystal polymer. The trace 1816 or circuit may take any pattern desired and may be selected to optimize the therapy. For example, the trace 1816 may be a continuous trace which winds back and forth over the surface of the liquid crystal polymer 1818. It is further contemplated that the peripheral shape of the shocking electrode 1808 may also be selected to reduce the defibrillation threshold. While the shocking electrode 1808 is illustrated as having a substantially oval peripheral shape, the shocking electrode 1808 may take any shape desired such as, but not limited to rectangular, circular, square, polygonal, tear drop, etc. The shocking electrode 1808 may have a larger surface area and/or shadow than a typical shocking coil electrode. It is contemplated that increasing the surface area and/or shadow may allow the defibrillation threshold to be lowered.

FIGS. 35B-35E show top view of alternative shocking electrodes 1808 that may be used with the illustrative lead and electrode assembly 1800 described above. The shocking electrodes 1808 illustrated in FIGS. 35A-35E should not be considered to be inclusive of all possible arrangements of the printed circuit patch but rather examples of some possible configurations. The configurations of printed traces 1816 and electrodes 1820 are endless and may be highly customized to achieve a desired defibrillation threshold. FIG. 35B illustrates a shocking electrode 1808 that includes a plurality of traces 1816 fanning out from a central area of the liquid crystal polymer 1818, in a similar manner to the veins of a leaf. A printed electrode 1820 may be positioned at the end of all or some of the traces 1816. The printed electrodes 1820 may vary in shape and size as desired.

FIG. 35C illustrates a shocking electrode 1808 that includes a plurality of traces 1816. Each trace 1816 may have a shape which mirrors the peripheral shape of the liquid crystal polymer 1818. The traces 1816 may be spaced a distance from one another at regular or irregular intervals. The traces 1816 may get progressively smaller towards the center of the liquid crystal polymer 1818. In some instances, the traces 1816 may generally resemble a loop-type fingerprint. The number and/or size of the traces 1816 may vary, as desired.

FIG. 35D illustrates a shocking electrode 1808 that include a centrally located electrode 1820 and a plurality of electrodes 1820 positioned about a perimeter of the liquid crystal polymer 1818. The electrodes 1820 may be connected through a series of traces 1816. The electrodes 1820 may vary in shape, size, and/or positioning as desired.

FIG. 35E a shocking electrode 1808 having a bulbous shape. The shocking electrode 1808 may include a plurality of electrodes 1820. In some instances, the electrodes 1820 may be sized and shaped to mirror a perimeter of the liquid crystal polymer 1818. The electrodes 1820 may be connected to one or more traces 1816. The electrodes 1820 may vary in shape, size, and/or positioning as desired.

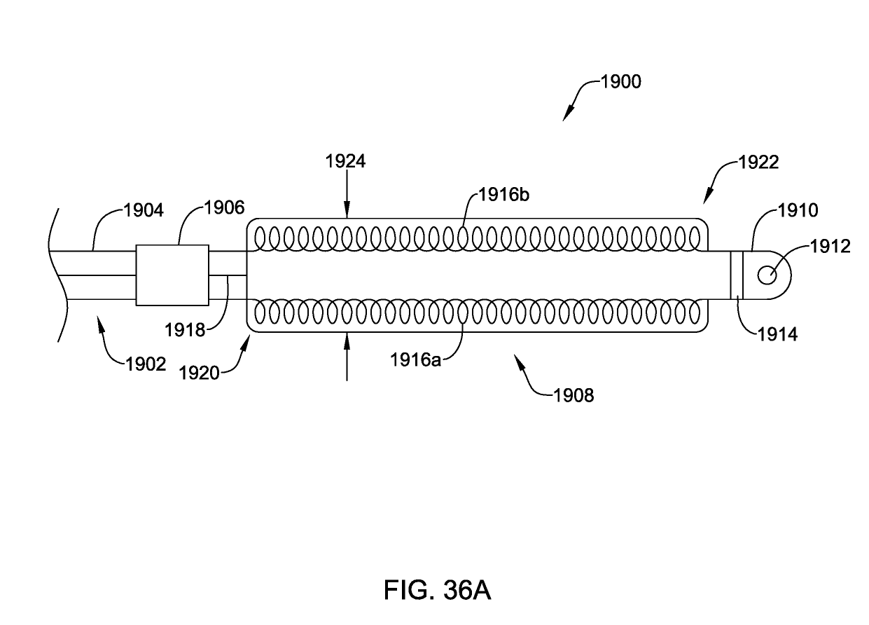

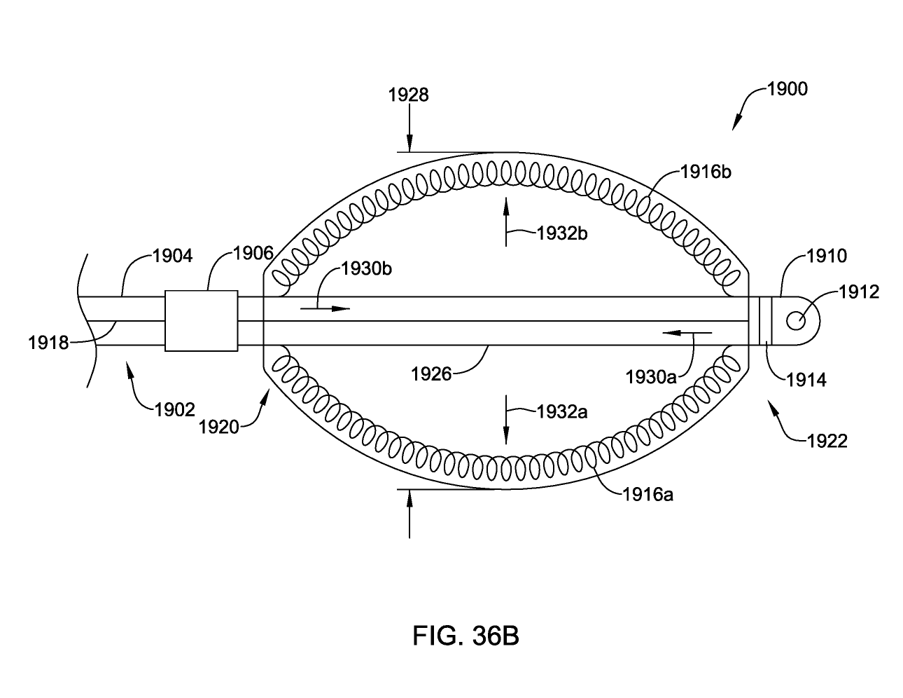

FIGS. 36A and 36B show a top view of another illustrative lead and electrode assembly 1900 for use with an implantable cardiac rhythm management system. In some embodiments, the illustrated assembly 1900 may be configured to move between a collapsed or delivery configuration, shown in FIG. 36A and an expanded or implanted configuration, shown in FIG. 36B. However, it is contemplated that the illustrative lead and electrode assembly 1900 of FIG. 36A may be both the delivery configuration and the implanted configuration. Similarly, the illustrative lead and electrode assembly 1900 of FIG. 36B may be both the delivery configuration and the implanted configuration.

The lead 1902 extends from a proximal end through an intermediate region 1904 to a distal end having a proximal electrode 1906, a coil electrode 1908, and a distal tip electrode 1910. The positioning and/or spacing of the electrodes 1906, 1908, 1910 may be adjusted and/or reconfigured to optimize sensing and/or therapy delivery. For example, both sensing electrodes 1906, 1910 may be placed proximal or distal to the coil electrode 1908. This is just an example.

The distal tip electrode 1910 is shown with a suture hole 1912. The suture hole 1912 may be coupled to a base portion 1914. Other designs may be used. In some embodiments, a suture hole 1912, or other fixation means, may not be required and/or may not be provided.

The coil electrode 1908 may be formed from two or more individual electrodes 1916a, 1916b. In some embodiments, the electrodes 1916a, 1916b may be coil electrodes. In other embodiments, the electrodes 1916a, 1916b may be other electrically active members, such as, but not limited to, struts. While the coil electrode 1908 is illustrated as including two electrodes 1916a, 1916b, the coil electrode 1908 may to including any number of individual electrodes desired, such as, but not limited to, one, two, three, four, five, or more. Further, in either configuration, coil electrodes 1916a, 1916b may be positioned close to one another (e.g. touching) or spaced a distance, as desired. The coil electrode 1908 may be affixed to the lead body 1902 at its proximal end 1920 and its distal end 1922. As shown in FIG. 36B, in some embodiments, the lead body 1902 may include a portion 1926 that extends between the proximal end 1920 and the distal end 1922 of the coil electrode 1908. It is contemplated that the lead body 1902 may include a telescoping feature or nested tubular members that allows the proximal end 1920 and/or distal end 1922 of the coil electrode 1908 to be moved along a longitudinal axis of the system 1900, such as in the direction of arrows 1930a, 1930b, shown in FIG. 36B. In other embodiments, the lead body 1902 may be disposed within one or both of the electrodes 1916a, 1916b. While not explicitly shown, the coil electrode 1908 may include a lumen or passageway for receiving a stylet or other delivery aid.

Each of the electrodes 1916a, 1916b may be formed from a round or flat (ribbon) wire, as desired. The wires may be relatively straight or coiled, as desired. In some instances, adjacent windings of the electrodes 1916a, 1916b may be in contact with one another while in other instances adjacent windings may be spread out or spaced a distance from one another, as desired. It is contemplated that the individual coil 1916a, 1916b may have the same or similar structure, or may be different, as desired. For example one electrode 1916a may be more tightly wound than the other 1916b. This is just an example.

The electrodes 1916a, 1916b may be actuatable or expandable from a delivery configuration having a first width 1924, shown in FIG. 36A, to an implanted configuration having a second larger width 1928, as shown in FIG. 36B. While the embodiments shown in FIGS. 36A and 36B are described as movable between two different configurations, it is contemplated the lead and electrode assembly 1900 may be fixed in either arrangement. In other words, in some embodiments the electrodes 1916a, 1916b may be movable relative to one another while in other embodiments, the electrodes 1916a, 1916b may be in a fixed arrangement relative to one another. It is contemplated that the coil electrode 1908, in either the delivery configuration or the implanted configuration, may be similar in size to the coil electrode 308 described above. The coil electrode 1908 may have a larger surface area and/or shadow than a typical shocking coil electrode. It is contemplated that increasing the surface area and/or shadow may allow the defibrillation threshold to be lowered which may allow the canister to have a smaller profile.

The lead and electrode assembly 1900 may be actuated between the delivery configuration and the implanted configuration using any number of deployment mechanisms. In one example, the distal electrode 1910 may be secured to the tissue. Once the distal end has been secured, the lead body 1902 may be distally advanced to apply a pushing force to the proximal end 1920 of the coil electrode 1908 using, for example a push-pull member 1918. This may cause the coil electrodes 1916a, 1916b to bias outward, for example in directions 1932a, 1932b, shown in FIG. 36B while also shortening in length, as shown at arrows 1930a, 1930b. It is contemplated that the same result may be achieved by applying a proximal, or pulling force to the distal end 1922 of the coil 1908 using the push-pull member 1918. In yet another example, the coil electrodes 1916a, 1916b may be formed in the expanded configuration illustrated in FIG. 36B. The coil electrodes 1916a, 1916b may be compressed into a lower profile delivery configuration through the application of a biasing force. For example, when the coil electrodes 1916a, 1916b are disposed within a delivery tool, the delivery tool may maintain the coil electrodes 1916a, 1916b in a reduced profile configuration.

Any of the lead and electrode assemblies described above may be configured to be self-expanding such that an actuation mechanism is not required. In other words, a delivery device may hold the lead and electrode assembly in a collapsed configuration and upon proximal retraction of the delivery device, the lead and electrode assembly assumes and expanded configuration.

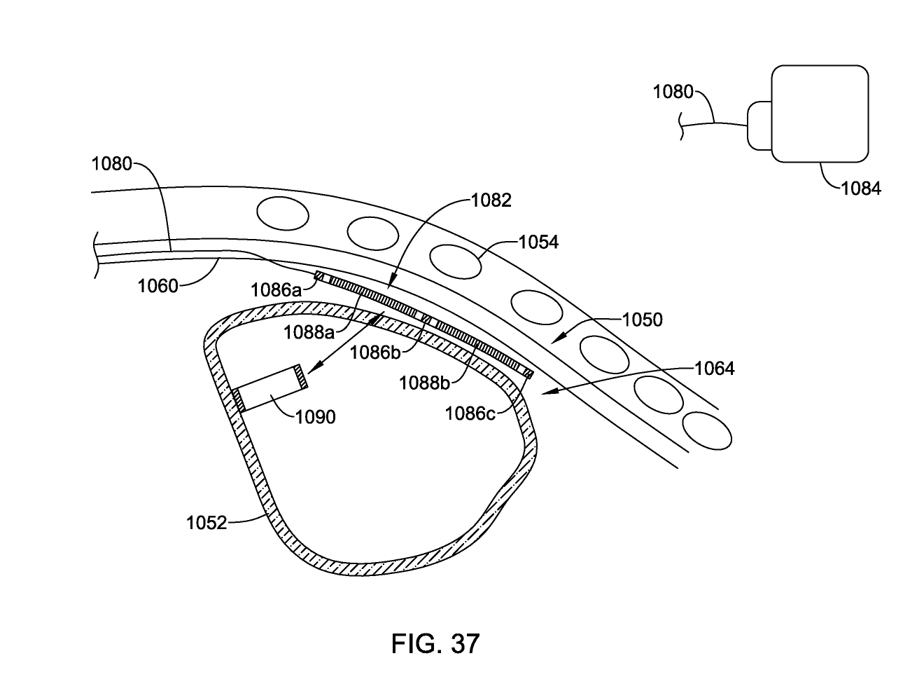

FIG. 37 is a lateral view of implantation of the implantable cardiac stimulus device of FIG. 25 in the mediastinal space 1064. In the example, the implantable cardiac stimulus device 1080, 1082, 1084 is shown concomitant with a leadless cardiac pacemaker (LCP) 1090. An illustrative LCP 1090 may include several functional blocks including a communications module, a pulse generator module, an electrical sensing module, and a mechanical sensing module. A processing module may receive data from and generate commands for outputs by the other modules. An energy storage module is may take the form of a rechargeable or non-rechargeable battery, or a supercapacitor, or any other suitable element.

Various details of the internal circuitry of an LCP 1090, which may include a microcontroller, microprocessor or a state-machine architecture, are further discussed in US PG Patent Publications 20150360036, titled SYSTEMS AND METHODS FOR RATE RESPONSIVE PACING WITH A LEADLESS CARDIAC PACEMAKER, 20150224320, titled MULTI-CHAMBER LEADLESS PACEMAKER SYSTEM WITH INTER-DEVICE COMMUNICATION, 20160089539, titled REFRACTORY AND BLANKING INTERVALS IN THE CONTEXT OF MULTI-SITE LEFT VENTRICULAR PACING, and 20160059025, titled, MEDICAL DEVICE WITH TRIGGERED BLANKING PERIOD, as well as other patent publications. Illustrative architectures may also resemble those found in the Micra™ (Medtronic) or Nanostim™ (St. Jude Medical) leadless pacemakers.

In this example, the ITV is shown at 1050 relative to the heart 1052 and ribs 1054. A device housing is shown at 1084 and couples to a lead 1080 which enters the superior epigastric vein and then passes first into the ITV 1050 and the, more superiorly, again exits the ITV into the mediastinum 1064, although other ways of accessing the ITV 1050 may be utilized as discussed herein. Such an exit from the ITV 1050 may be accomplished by advancing a guidewire through the vein wall, and then passing a dilator/guide catheter over the guidewire and through the vessel wall, with the lead then being introduced through the guide catheter that has passed through the vessel wall, after removing the guidewire in a manner similar to that described with respect FIGS. 21-25. This allows the distal portion of lead 1080 to reside in the mediastinum 1064 and somewhat closer to the heart 1052.

The lead 1080 is shown having a plurality of electrodes including those at 1086, 1088. An LCP is shown in a ventricle at 1090. The LCP can thus communicate with the mediastinium pacing system using, for example conducted communication with a pair of any of the lead electrodes 1086, 1088, or, if desired, a different combination of electrical contacts such as a conductive element or portion of the housing of the device 1084 paired with one of the electrodes 1086, 1088. It is contemplated that the LCP 1090 may communicate with any of the lead/electrode arrangements and/or pacing systems described herein. For example, a communication may be issued using at least one of the lead electrodes, and received by electrodes of the LCP.

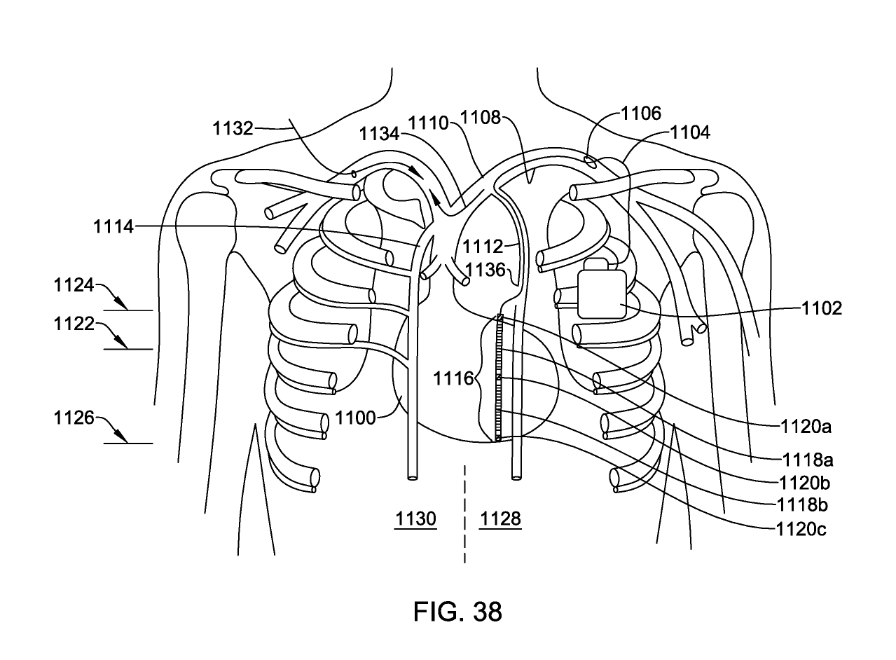

FIG. 38 shows implantation of an implantable cardiac stimulus system with the lead in the mediastinum using a superior access approach. The system includes an implantable pulse generator 1102 which may be placed in the subclavicular location shown (or any other suitable position, as desired). A lead 1104 passes into the venous access point 1106 into the subclavian vein 1108 and to the brachiocephalic vein 1110. The lead then enters the left ITV 1112. For such an introduction, in one example, a guide catheter can be used to direct the lead 1104 through the ostium of the chosen ITV, with or without use of a guidewire.

In some examples, a flexible lead is used having a lumen therein to receive a guidewire or stylet to enhance pushability through the valves of the ITV 1112. In another example, a flexible lead may be introduced with the support of the guide catheter during advancement. In this latter example, the guide catheter may receive the lead 1104 through a guide catheter lumen that serves to retain a fixation apparatus or shape for the flexible lead, such as a 2-dimensional or 3-dimensional curvature (see FIGS. 10-11), tines (see FIG. 12), an expandable member (see FIG. 15), or hooks or a side-extending engagement structure (see FIG. 16).

In another alternative, the guide catheter and guidewire may be omitted by providing a lead with a flexible or steerable structure, and/or a lead configured for implantation using a steerable stylet. For example, a lead may be configured to be implanted using a steerable stylet in a lumen thereof, with the initial placement into the ostium of the left ITV 1112 (or right ITV 1114, if desired) at the distal end of the introducer sheath, possibly using contrast visualization, if desired. Once initial access is achieved, simply pushing the stylet should be sufficient to implant the lead to a desired level in the ITV. The stylet may have a secondary function of preventing an anchoring structure of the lead from assuming an anchoring shape or releasing an anchoring tine, hook, expandable member, stent or other device.

The lead 1104 may exit the left ITV 1112 at an exit location 1136. The lead 1104 may be guide into the mediastinum using any of the methods described above with respect to FIGS. 21-26. The lead 1104 may be positioned between the heart 1100 and the left ITV 1112. In the example, the lead 1104 includes a multi-electrode distal structure as shown at 1116. The structure includes a proximal coil 1118A separate from a distal coil 1118B. The coils 1118A/B and canister 1102 may serve as therapy delivery electrodes. As such there may be multiple therapy vectors such as between coil 1118A and coil 1118B, between either of coils 1118A and 1118B and the canister 1102, or between a combination of two of the three therapy electrodes 1118A, 1118B and canister 1102, and the third such electrode, such as by linking coils 1118A and 1118B in common as the anode or cathode relative to the canister 1102.

A plurality of ring electrodes may be provided as shown at 1120A, 1120B, and 1120C. Electrode 1120C may also or instead be a tip electrode. Electrodes 1120A/B/C may serve as sensing electrodes. The coils 1118A, 1118B may also serve as sensing electrodes. In addition, one or more of the ring or tip electrodes 1120A, 1120B, 1120C may be used for therapy delivery. In an example, defibrillation therapy may use coils 1118A, 1118B coupled in common as the opposing pole to the canister 1102, while pacing therapy may use coils 1118A and 1120B as opposing electrodes for post-shock pacing therapy, with a still different combination of electrodes used to provide ventricular pacing therapy for example by pacing between coil 1118B and tip electrode 1120C.

Line 1122 is provided, illustratively, to separate the atria and ventricles. The lead 1104 may be placed as shown such that the proximal coil 1118A is about level with the atria, and distal coil 1118B is about level with the ventricles, if desired. In some examples fewer or different electrodes may be provided on the lead 1104 such as by excluding one or the other of the proximal coil 1118A or distal coil 1118B. Various designs are also shown herein.

Line 1124 is provided to indicate the top of the heart, with the apex or bottom of the heart marked at 1126. In some examples, one or more electrodes on the lead 1104 are provided at or inferior to the apex 1126, or at or superior to the top 1124 of the heart. In the example shown, on the other hand, the electrodes are located generally between the apex 1126 and top 1124 of the heart.

The illustration shown in FIG. 38 places the lead on the left side 1128 of the patient. In other examples, the right side 1130 of the patient may instead or in addition be accessed, including the right ITV 1114. Access to the right ITV 1114 may be achieved by advancing a guide catheter and/or guidewire from the left subclavian access 1106 as shown by arrow 1134 across to the ostium of the right ITV 1114.

Alternatively, access to the right ITV may be achieved as shown at arrow 1132 by entering the right subclavian vein in a mirror image procedure of that shown in FIG. 4. In some examples, each of the left and right ITV 1112, 1114 may be used to place a lead 1104 in the mediastinum. Pacing between right and left side lead placements may be performed to target specific chambers or chamber combinations, or sensing may be performed using one pair of electrodes with therapy delivery using a different pair of electrodes to achieve resynchronization or other desirable effect.

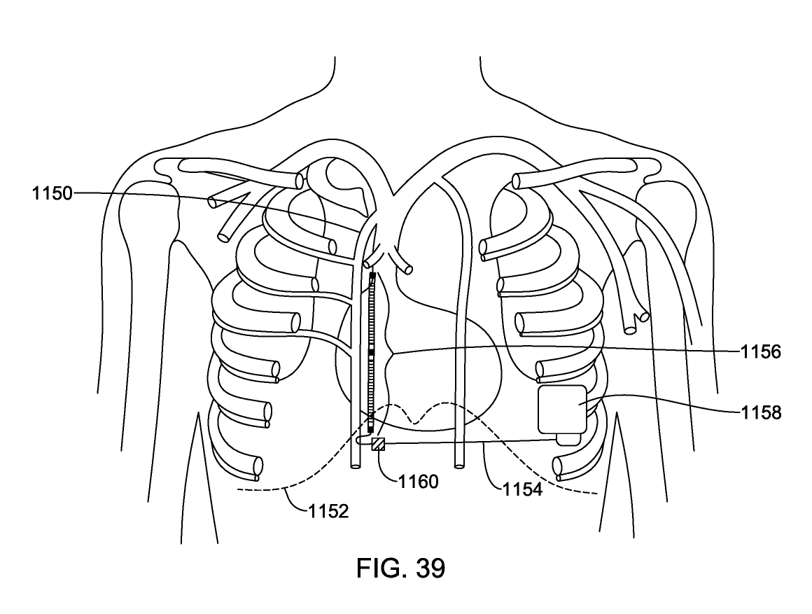

FIG. 39 shows implantation of an implantable cardiac stimulus system with the lead in the mediastinum using an inferior access approach. In order to access either the left superior epigastric vein or the right superior epigastric vein (see, for example, FIG. 6A), a physician may palpate for the xiphoid process and then use ultrasound guided access to obtain needle entry into the desired vein on the desired side of the xiphoid. This inferior approach preserves the upper thoracic vasculature in the event that the patient later needs a traditional transvenous, intracardiac system, or for use in other procedures. Such access may also reduce the potential for lead fracture such as that caused by subclavian crush. Once access to a selected superior epigastric vein is achieved, the vessel can be traversed in a superior direction to place the lead at a desired level by entering the corresponding ITV.

The access may generally resemble the well-known Seldinger technique, with an initial needle puncture using a hollow needle or trocar. A guidewire is passed through the hollow needle or trocar, which can then be removed. An introducer sheath, typically having a dilator therein and a valve at a proximal end thereof, is then inserted over the guidewire and into the desired blood vessel. The dilator and/or guidewire can then be removed, leaving in place the valved introducer sheath to allow introduction of interventional devices and/or a lead therethrough. At the conclusion of the lead implantation procedure, a sealing device such as a suture sleeve can be placed to seal the puncture site to the implantable lead left therein. The aim may be to access the ITV or superior epigastric vein at or near the 7^{th} rib margin in a window adjacent to the xiphoid process that may be described as a paraxiphoid window.

In another example, a cut-down technique may be used to access the desired vein by incision through the skin. Next, possibly after visual confirmation the desired vessel is accessed, incision into the selected vein can be made. In another example, anatomical landmarks such as the rib margin and/or infrasternal angle may be used to facilitate venipuncture into the desired vein.

In the example shown in FIG. 39, the right ITV 1150 has been accessed by introduction through the superior epigastric vein from a location inferior to the rib margin 1152. An implantable device has been placed including a lead 1154 having a distal electrode structure 1156 and a canister 1158, with the canister 1158 placed at approximately the left axilla. The canister 1158 may be placed as desired, for example at the anterior axillary line, the midaxillary line, or in the posterior axillary line. The lead 1154 and/or distal electrode structure 1156 may exit the right ITV 1150 in a manner similar to that described with respect to FIGS. 21-26.

In the illustration, a suture sleeve is shown at 1160 and is used to fixate the lead 1154, for example, to the subcutaneous fascia. For placement, the right ITV 1150 is accessed as described above, and a tunnel is established between the left axilla and the access location such as along a portion of the inframammary crease. The lead 1154 may, in this case, be relatively stiff to assist in keeping it emplaced in the patient as shown, if desired. Various designs are shown herein for the lead as well, including tines, hooks, curvature or bias of the lead, and inflatable or expandable structures. In the example of FIG. 39, a left axillary canister location is shown; a right sided, pectoral or subclavicular left or right position may be used instead, in combination with the right ITV placement 1150 or, alternatively a left ITV placement.

During implantation, a sheath may be provided over the lead 1154, or at least a portion thereof, to retain or restrain a fixation apparatus or shape for the flexible lead, such as a 2 or 3 dimensional curvature (see FIGS. 10-11), tines (see FIG. 12), an expandable member (see FIG. 15), or hooks or a side-extending engagement structure (see FIG. 16). A stylet may be placed through the lead 1154, or a portion thereof, to retain a straight shape during implantation; upon removal of the stylet, a curvature (see FIGS. 10-11) may then be released for securing the lead 1154 in place.

The lead 1154 may include additional or different electrodes than those shown. For example, another coil electrode may be placed on a more proximal portion of the lead 1154 to reside along the inframammary crease in a location between the canister 1158 and the point of access into the superior epigastric vein. The additional coil at this location may be used for defibrillation or other therapy purposes, or for sensing. If desired, second or more leads may also be placed.

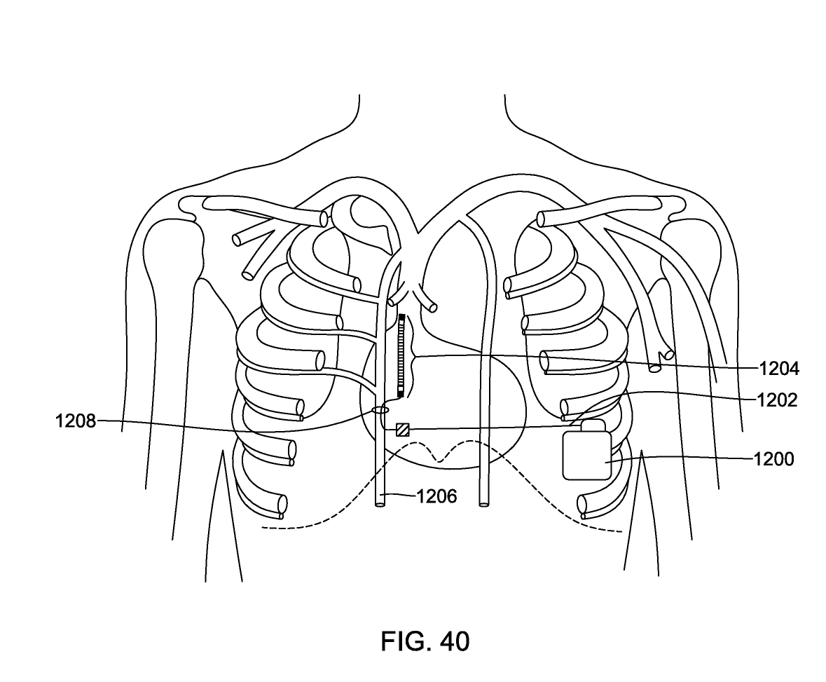

FIG. 40 shows implantation of an implantable cardiac stimulus system with the lead in the mediastinum using an intercostal approach. Any intercostal space overlying either of the right and left ITV may be a suitable point of entry, however, more superior or inferior positions may be preferred to allow passage of the distal end of a lead along a significant region of the ventricles and atria by passing in a single direction. Access may be had using ultrasound guided needle insertion. Again, the access method may resemble the Seldinger technique, though in this case the muscle in the intercostal space would first be traversed. A needle may be used to establish puncture using ultrasound guidance, with a guidewire passed therethrough. Once the puncture is made and the guidewire is in the desired blood vessel, the needle is removed, keeping the guidewire in place, and an appropriately sized introducer sheath (optionally including a dilator) is placed over the guidewire.

The alternative in FIG. 40 allows access from either superior or inferior positions while preserving the upper thoracic vasculature. An advantage over the approach of FIG. 39 is that the use of a suture sleeve attachment with FIG. 40 would occur on the fascia over the ribcage near the intercostal access point, making suture sleeve use easier and avoiding movement between the point of venous system entry and the point of fixation. On the other hand, a user may be more comfortable accessing the veins at a location where the ribs and intercostal muscles do not interfere; thus, each of the various approaches herein has advantages and disadvantages relative to one another.

In this example, an implantable system having an implantable pulse generator 1200 and lead 1202 with distal electrode structure 1204 has been emplaced in a patient in the mediastinum. The lead 1202 and/or distal electrode structure 1204 may exit the right ITV 1206 in a manner similar to that described with respect to FIGS. 21-26. The right ITV 1206 is accessed using an intercostal access point at 1208. Such an access position may be labeled a parasternal access position.

The access 1208 may be achieved by inserting a needle, preferably under guidance such as by the use of an ultrasound guided needle, into a chosen intercostal space, preferably low on the ribcage and near the sternum, through the muscle of the intercostal space and into the right ITV 1206. A guidewire can be passed through the needle and an introducer sheath passed over the guidewire after removal of the needle. Other techniques may be used instead, and other access points may be selected.

A suture sleeve may be used to secure the lead 1202 over the ribcage as desired. The lead 1202, as with all other implanted leads shown herein, may include a fixation structure such as bends or curves along its distal length, or tines, hooks or expandable members at its distal end to secure its position within the ITV 1206.

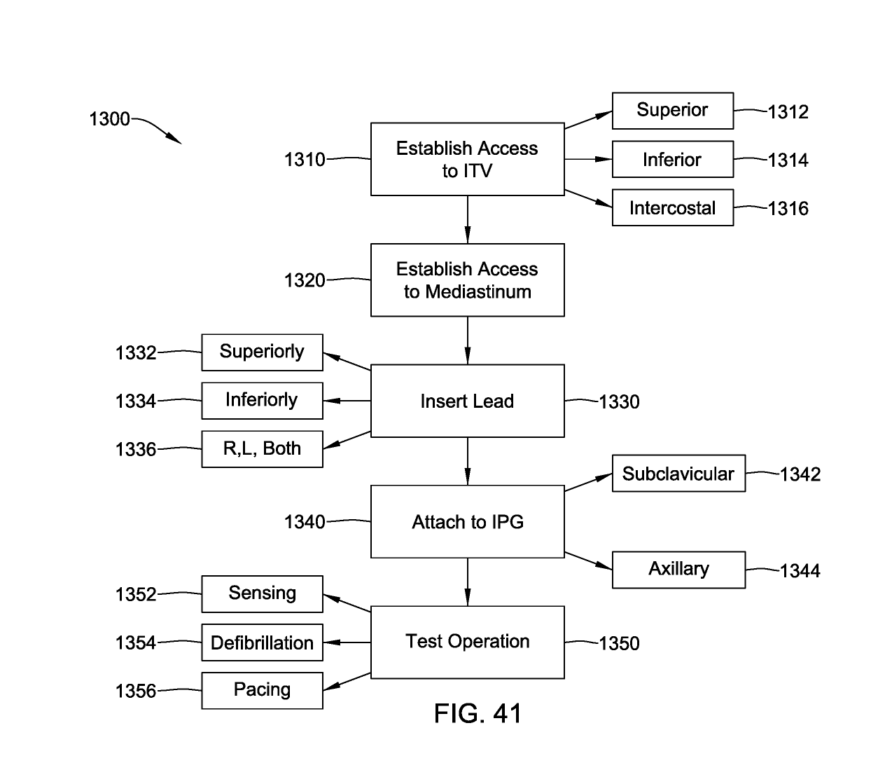

FIG. 41 is a block flow diagram for an illustrative method for providing a cardiac stimulus system to a patient. As shown at 1300, the method comprises establishing access to the ITV 1310, establishing access to the mediastinum 1320, inserting a lead in the mediastinum 1330, attaching an IPG to the lead 1340, and performing test operations 1350.

For example, establishing access to the ITV 1310 may include accessing from a superior position 1312 such as by entering the subclavian vein and passing through the ostium of the ITV in the brachiocephalic vein. In another example, establishing access to the ITV 1310 may include accessing from an inferior position 1314 such as by entering the superior epigastric vein and passing superiorly therefrom into the ITV. In some examples, access via locations 1312, and 1314 may include accessing via a second blood vessel such as by accessing superiorly 1312 by way of the subclavicular vein and brachiocephalic vein, or accessing inferiorly 1314 through the superior epigastric vein. In still another example, establishing access to the ITV may include accessing in an intercostal space 1316 such as by penetrating an intercostal space and entering the ITV using a Seldinger technique.

Establishing access to the mediastinum 1320 may include placing a sheath in the ITV, puncturing the ITV with a needle, placing a first guidewire, placing a second sheath and dilator set over the first guidewire, retracting the first guidewire and dilator, and placing a second guidewire. In some cases, an expandable balloon may be advanced over the second guidewire. The expandable balloon may be expanded to gently displace some the tissues within the mediastinum to create a cavity for placement of a lead and electrode assembly. In some cases, access to the mediastinum 1320 may be established using fewer medical devices. For example, one or more of the sheaths, guidewires, needles, and/or dilators may not be required to access the mediastinum.

In an example, inserting a lead 1330 may include insertion superiorly 1332, such as by starting in an inferior position 1312 inferior to the lower rib margin or intercostally 1316 from an inferior intercostal location, and advancing the lead in a superior direction. For another example, inserting a lead 1330 may include insertion inferiorly 1334, that is starting at a superior location 1314 or at a superior intercostal location 1316, and advancing the lead in an inferior direction. In either such example, the right ITV, left ITV, or both ITV vessels may be used to place a lead in the mediastinum, as indicated at 1336.

During the implantation procedures, contrast or other visualization may be used in various ways. For example, when using a superior access 1312 to the ITV, entering for example via the brachiocephalic vein, contrast or other visualization may be used to track the position of a guidewire, guide catheter or the lead itself into the ostium and then down in to the ITV. In addition, regardless the access route to the ITV, the step of establishing access to the mediastinum may include use of visualization to observe the exit from the ITV and into the mediastinum. Lateral X-ray or other visualization may be used as well to observe lead positioning both in terms of how superior/inferior the lead and its electrodes are, as well as whether the lead is deep enough or shallow enough, as the case may be, in the mediastinum to achieve therapy and/or anchoring aims, and to avoid piercing or poking the lung and/or pericardium, if desired.

Other vessels and implanted lead locations may also be used (such as having a lead in the right ITV, left ITV, both ITVs, azygos vein, an intracardiac lead, a subcutaneous lead) or additional devices such as a separately implanted leadless cardiac pacemaker may be included as well. In a further example, one or more of the transverse veins that flow into the ITV may be used for placement of an electrode or lead. For example, upon accessing an ITV, a physician may further access and emplace a lead or electrode into one of the anterior intercostal veins which run along the intercostal spaces of the anterior chest.

In an example, attaching to an IPG may include attaching to a canister located in a subclavicular location 1342, historically a common place to put an implanted canister for a transvenous defibrillator or pacemaker. In another example, attaching to an IPG may include attaching to a canister located in an axillary position 1344, such as that used with the S-ICD System. Other IPG locations may be used. Attachment may be directly to the IPG or to a splitter, yoke, or lead extension, if desired.

In an example, test operation 1350 may be used to verify one or both of device functionality and efficacy. For example, sensing operations 1352 may be tested and configured to check for adequate signal availability, for example, or by setting gain, filtering, or sensing vector selection parameters. Defibrillation operations 1354 may be tested by inducting an arrhythmia such as a ventricular fibrillation to determine whether the device will sense the arrhythmia and, if the arrhythmia is sensed, to ensure that the device can adequately provide therapy output by delivering defibrillation at a preset energy. Defibrillation testing 1354 may include determining for a given patient an appropriate defibrillation threshold, and setting a parameter for therapy delivery at some safety margin above the defibrillation threshold.

Prior transvenous systems would typically deliver up to 35 Joules of energy at most, with storage of up to 40 Joules of energy, using peak voltages in the range of up to nearly 1300 volts. The S-ICD System can deliver up to 80 Joules of energy, with 65 Joules often used for in-clinic system testing, with a peak voltage in the range of 1500 volts. The ITV location may facilitate energy levels similar to those of traditional transvenous systems (5-35 Joules, approximately), or may be somewhat higher (5 to about 50 joules, for example), or may still be higher (10 to about 60 joules, for example). Pacing thresholds may also be closer to those for traditional transvenous systems than the more recent S-ICD System.

In an example, pacing testing operation 1356 may include determining which, if any, available pacing vectors are effective to provide pacing capture. If desired, parameters may be tested as well to determine and optimize settings for delivery of cardiac resynchronization therapy. This may include testing of pacing thresholds to optimize energy usage and delivery, as well as checking that adverse secondary effects, such as patient sensation of the delivered pacing or inadvertent stimulation of the phrenic nerve, diaphragm or skeletal muscles are avoided.

In some cases, the left and/or right ITV may be used to access the mediastinum. The target location in region generally contains some loose connective tissues, muscle, nerves and blood vessels. Anchoring a lead may be desirable, for example, in the region between the left and/or right ITV (and beneath the rib cage) and a lateral side of the heart. From such a position, beneath the rib cage, the amount of energy required for defibrillation and pacing efficacy would logically be lower than outside of the sternum and/or rib cage, since the mediastinum location is closer to the heart and bone is generally not a very good conductor of electrical energy, at least when speaking in terms of the tissues in the human body. However, tunneling in this region is not so necessary as it may be in other locations, particularly the subcutaneous space, where the innermost layers of dermis must be separated from underlying muscle, connective tissue and fascia. Indeed, the insertion of a lead through the ITV (e.g., using any of superior access, inferior access, and/or intercostal access) may enable safe placement in the mediastinum.

In one example, the musculophrenic vein may be used. The musculophrenic vein runs along the lower rib margin and may be accessed in a manner that will be termed, for purposes herein, as an inferior access location as it would be inferior to the lowest rib. The musculophrenic vein and superior epigastric vein come together at the lowest end of the internal thoracic vein. Due to its adjacency to a bony structure (the costal margin), the musculophrenic vein may be useful as its access may be simpler than that of the superior epigastric vein (as the position can be readily ascertained) or the internal thoracic vein (as access would not require going through an intercostal).

In any of the above examples, additional lead placement may take place (e.g., in addition to an ITV lead and/or a mediastinal lead). For example, an additional lead may be placed subcutaneously, within the heart, or in a different blood vessel such as the azygos vein. Additional device placement may occur as well, including, for example, the placement of a leadless cardiac pacemaker in one or more chambers of the heart.

The above examples facilitate a number of therapy options. For example, defibrillation therapy may be delivered in various configurations such as, without limitation:
Between a left ITV (and/or mediastinal) electrode or combination of electrodes and a right ITV (and/or mediastinal) electrode or combination of electrodes;
Between a left ITV (and/or mediastinal) electrode and a device housing placed in the left axilla or left subclavicular location;
Between a right ITV (and/or mediastinal) electrode and a device housing placed in the left axilla or left subclavicular location;
Between a left ITV (and/or mediastinal) electrode and a device housing placed in the right axilla or right subclavicular location;
Between left and right ITV (and/or mediastinal) electrodes electrically in common and a right or left axillary or subclavicular canister.
Between one ITV (and/or mediastinal) electrode and a second ITV (and/or mediastinal) electrode in common with a device canister in the left or right axilla or subclavicular location
Between a first electrode on a lead, and a second electrode on the same lead, where the first and second electrodes are in the same ITV (and/or mediastinal)
Between a first electrode on a lead, and a second electrode on the same lead, where the first electrode is in an ITV (and/or mediastinal), and the second electrode is in a tunnel leading to access to the ITV, such as in the inframammary crease on lead 410 in FIG. 7

In these examples, a “left ITV (and/or mediastinal) electrode” or “right ITV (and/or mediastinal) electrode” may include a single coil electrode or a combination of plural coils and/or one or more coils with one or more ring electrodes electrically in common. The above combinations may also be used for delivery of a bradycardia pacing therapy or an anti-tachyarrhythmia pacing therapy.

Further examples may provide a resynchronization therapy by delivering pacing pulses in various configurations, such as, without limitation:
In bipolar fashion within the left ITV (and/or mediastinal) to pace the left ventricle, and also in bipolar fashion within the right ITV (and/or mediastinal) to pace the right ventricle, with relative timing between the two sets of pacing therapies determined according to analysis of cardiac output or electrical response.
In bipolar fashion within one of the left or right ITV (and/or mediastinal) to stimulate a respective left or right ventricle in response to atrial sensed signals sensed with electrodes placed in an ITV (and/or mediastinal) at a superior location level with the atria.
In monopolar fashion between a device housing and one or both of left or right ITV (and/or mediastinal) electrodes, using for timing information atrial signals sensed using additional electrodes in at least one ITV (and/or mediastinal) and/or far-field sensed morphology detected using a device housing.

In an example, a heart failure or resynchronization therapy may be delivered as follows, with reference to FIG. 7. A pacing therapy may be delivered by sensing atrial activity using the distal two ring electrodes shown in the electrode assembly 412 to determine timing for pace therapy delivery using the proximal coil electrode and canister 414. Numerous other combinations may be had as can be seen to those skilled in the art.

Some embodiments of the present invention may take the form of an implantation tool set configured for use in implanting a cardiac device, such as a lead, into an ITV. Some such embodiments may include an introducer sheath. Some such embodiments may include a guide catheter. Some such embodiments may include a guidewire. Some such embodiments may further include a tool set for performing a Seldinger technique to access a blood vessel percutaneously.

Some embodiments of the present invention take the form of an implantable cardiac stimulus device comprising a lead and an implantable canister for coupling to the lead, the implantable canister housing operational circuitry configured to deliver output therapy in the form of at least one of bradycardia pacing, anti-tachycardia pacing, cardiac resynchronization therapy, or defibrillation, using a lead implanted in an ITV and a canister implanted in a patient.

As used herein, a coil electrode may be a helically wound element, filament, or strand. The filament forming the coil may have a generally round or a generally flat (e.g. rectangular) cross-sectional shape, as desired. However, other cross-sectional shapes may be used. The coil electrode may have a closed pitch, or in other words, adjacent windings may contact one another. Alternatively, the coil electrode may have an open pitch such that adjacent windings are spaced a distance from one another. The pitch may be uniform or varied along a length of the coil electrode. A varied pitch may be gradual tapered changes in pitch or abrupt or step-wise changes in pitch.

A coil electrode may have a length L that is generally larger than a width W. Round, oval or flattened coil electrodes may be used. Coil electrodes may have a length in the range of one to ten centimeters. In an example, a coil having a six or eight centimeter length may be used. In another example, a lead may have two four centimeter coils. Coils and leads may be in the range of four to ten French, or larger or smaller, in outer profile.

Coils and leads may be coated. For example, a thin permeable membrane may be positioned over a shock coil or other electrode and/or other portions of the lead to inhibit or to promote tissue ingrowth. Coatings, such as, but not limited to expanded polytetrafluoroethylene (ePTFE) may also be applied to the coil and/or lead to facilitate extraction and/or to reduce tissue ingrowth. In some embodiments, one or more of the electrodes, whether coils, rings, or segmented electrodes, include a high capacitive coating such as, but not limited to iridium oxide (IrOx), titanium nitride (TiN), or other “fractal” coatings which may be used, for example, to improve electrical performance. Steroidal and antimicrobial coatings may be provided as well.

The various components of the devices/systems disclosed herein may include a metal, metal alloy, polymer, a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers for use in the leads discussed above may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like.

In at least some embodiments, portions or all of the accessory devices and their related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the accessory devices and their related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the accessory devices and their related components to achieve the same result.

Any guidewire, introducer sheath, and/or guide catheter design suitable for medical interventions may be used for accessing the venous structures discussed herein.

The implantable systems shown above may include an implantable pulse generator (IPG) adapted for use in a cardiac therapy system. The IPG may include a hermetically sealed canister that houses the operational circuitry of the system. The operational circuitry may include various elements such as a battery, and one or more of low-power and high-power circuitry. Low-power circuitry may be used for sensing cardiac signals including filtering, amplifying and digitizing sensed data. Low-power circuitry may also be used for certain cardiac therapy outputs such as pacing output, as well as an annunciator, such as a beeper or buzzer, telemetry circuitry for RF, conducted or inductive communication (or, alternatively, infrared, sonic and/or cellular) for use with a non-implanted programmer or communicator. The operational circuitry may also comprise memory and logic circuitry that will typically couple with one another via a control module which may include a controller or processor. High power circuitry such as high power capacitors, a charger, and an output circuit such as an H-bridge having high power switches may also be provided for delivering, for example, defibrillation therapy. Other circuitry and actuators may be included such as an accelerometer or thermistor to detected changes in patient position or temperature for various purposes, output actuators for delivering a therapeutic substance such as a drug, insulin or insulin replacement, for example.

Some illustrative examples for hardware, leads and the like for implantable defibrillators may be found in commercially available systems such as the Boston Scientific Teligen™ ICD and Emblem S-ICD™ System, Medtronic Concerto™ and Virtuoso™ systems, and St. Jude Medical Promote™ RF and Current™ RF systems, as well as the leads provided for use with such systems.

Animal testing has been performed in the porcine model to illustrate feasibility. Such testing made use of selected leads including a prototype lead resembling that shown above in FIG. 10 having a coil electrode 612 with a length of about 4 centimeters, replacing tip electrode 614 with an atraumatic tip, and including two proximal ring electrodes 606, 608 for defibrillation testing between a canister emulator and the 4 cm coil showing at least a thirty-percent reduction in defibrillation threshold relative to a subcutaneous-only defibrillation test in the same animal, using the right ITV to left-sided canister. The prototype lead included a three-dimensional curvature for fixation purposes resembling a spiral.

Additional testing in the same animal made use of an Acuity™ X4 lead (Boston Scientific) for pacing purposes in a unipolar configuration, with the pacing also successful. Still further testing using a now obsolete Perimeter™ CS lead (Boston Scientific), with defibrillation testing also showing a significant reduction in threshold therapy energy. It is estimated that a reduction in defibrillation threshold was in the range of 30-50% for this animal relative to the subcutaneous defibrillation threshold. Each of these non-limiting examples can stand on its own, or can be combined in various permutations or combinations with one or more of the other examples.

In a first example, a method of implanting a lead for use in a cardiac stimulus system in a patient, the lead having at least one electrode thereon may comprise accessing the mediastinum by entering the internal thoracic vein (ITV) and then exiting the ITV to enter the mediastinum, creating a recess in the mediastinum, and inserting the lead into the recess created in the mediastinum to a desired location relative to the heart of a patient.

Alternatively or additionally to any of the examples above, in another example, the method may further comprise establishing access to a brachiocephalic vein of the patient and advancing a distal portion of the lead through the ostium of the ITV from the brachiocephalic vein.

Alternatively or additionally to any of the examples above, in another example, the step of establishing access to the brachiocephalic vein may comprise inserting an introducer sheath into one of the axillary, jugular, cephalic or subclavian veins of the patient and advancing at least the lead through the introducer sheath, into the brachiocephalic vein, and then through the ostium of the ITV.

Alternatively or additionally to any of the examples above, in another example, the method may further comprise advancing a guidewire to and into the ostium of the ITV.

Alternatively or additionally to any of the examples above, in another example, the method may further comprise advancing a guide catheter to and into the ostium of the ITV.

Alternatively or additionally to any of the examples above, in another example, the method may further comprise advancing a needle into the ITV.

Alternatively or additionally to any of the examples above, in another example, the method may further comprise puncturing a wall of the ITV.

Alternatively or additionally to any of the examples above, in another example, the method may further comprise advancing a dilator and inner sheath set through the puncture in the wall of the ITV.

Alternatively or additionally to any of the examples above, in another example, at least one of the dilator or the inner sheath may be deflectable.

Alternatively or additionally to any of the examples above, in another example, the dilator or the inner sheath may displace a region of tissues in the mediastinum to create the recess.

Alternatively or additionally to any of the examples above, in another example, the method may further comprise removing the dilator and the guidewire.

Alternatively or additionally to any of the examples above, in another example, the method may further comprise advancing a second guidewire through the puncture in the wall of the ITV to the desired location in the mediastinum.

Alternatively or additionally to any of the examples above, in another example, the method may further comprise advancing a balloon catheter having an expandable balloon over the second guidewire to position the expandable balloon adjacent to the desired location.

Alternatively or additionally to any of the examples above, in another example, the method may further comprise expanding the expandable balloon to compress a region of tissues in the mediastinum to create the recess.

Alternatively or additionally to any of the examples above, in another example, the step of inserting the lead may comprise advancing the lead over the second guidewire.

Alternatively or additionally to any of the examples above, in another example, the step of inserting the lead may comprise advancing the lead through a delivery device.

Alternatively or additionally to any of the examples above, in another example, the method may further comprise establishing access to the ITV through an intercostal space between two ribs which may include inserting a needle into one of the ITV through the intercostal space and advancing a sheath into the intercostal space and into the ITV. The step of inserting the lead may comprise advancing the distal end of the lead through the sheath and into the ITV.

Alternatively or additionally to any of the examples above, in another example, the method may further comprise creating a puncture in a wall of the ITV and advancing the distal end of the lead through the puncture and into the mediastinum.

Alternatively or additionally to any of the examples above, in another example, the step of advancing the distal end of the lead through the sheath and into the ITV may comprise advancing the distal end of the lead in an inferior direction into the ITV.

Alternatively or additionally to any of the examples above, in another example, the step of advancing the distal end of the lead through the sheath and into the ITV may comprise advancing the distal end of the lead in a superior direction.

Alternatively or additionally to any of the examples above, in another example, the method may further comprise tunneling from the left axilla to the intercostal space, attaching an implantable pulse generator to the lead and implanting the pulse generator at the left axilla.

Alternatively or additionally to any of the examples above, in another example, the method may further comprise establishing access to the superior epigastric vein at a location inferior to the lower rib margin and introducing the lead through the epigastric vein and superiorly into the ITV.

Alternatively or additionally to any of the examples above, in another example, the step of establishing access to the superior epigastric vein may comprise inserting a needle into the superior epigastric vein, and advancing a sheath into the superior epigastric vein. The step of introducing the lead through the superior epigastric vein may comprise advancing the distal end of the lead through the sheath and into the ITV.

Alternatively or additionally to any of the examples above, in another example, the method may further comprise creating a puncture in a wall of the ITV and advancing the distal end of the lead through the puncture and into the mediastinum

Alternatively or additionally to any of the examples above, in another example, the method may further comprise tunneling from the left axilla to the location where the ITV is accessed and a proximal portion of the lead in the tunnel, wherein the method may further comprise attaching an implantable pulse generator to the lead and implanting the pulse generator at the left axilla.

Alternatively or additionally to any of the examples above, in another example, the method may further comprise anchoring the lead in the ITV and/or mediastinum using an inflatable balloon.

Alternatively or additionally to any of the examples above, in another example, the method may further comprise anchoring the lead in the ITV and/or mediastinum using an expandable member, the expandable member selected from the group consisting of a lobe, a tine, a hook, or a stent.

Alternatively or additionally to any of the examples above, in another example, the lead may be configured to have a curvature and the method may further comprise anchoring the lead by allowing it to assume the curvature once inserted into the ITV and/or mediastinum.

Alternatively or additionally to any of the examples above, in another example, the method may further comprise attaching a suture sleeve and suturing the suture sleeve to subcutaneous tissue to the lead to hold the lead in position.

Alternatively or additionally to any of the examples above, in another example, the ITV may be the right ITV.

Alternatively or additionally to any of the examples above, in another example, the ITV may be the left ITV.

Alternatively or additionally to any of the examples above, in another example, the lead may comprise a first collapsed configuration and a second expanded configuration.

In another example, a method of implanting a cardiac stimulus system may comprise performing the method of any of the examples described herein to implant a first lead in a first recess in the mediastinum adjacent to the right ITV, performing the method of any of the examples described herein to implant a second lead in a second recess in the mediastinum adjacent to the left ITV, and coupling the first and second leads to a pulse generator for the cardiac stimulus system.

In another example, a method of treating a patient may comprise delivering therapy between a first electrode disposed on a lead which is placed in a recess created in a mediastinum of a patient through an ITV, and at least a second electrode.

Alternatively or additionally to any of the examples above, in another example, the therapy may be a defibrillation therapy, and the second electrode is disposed on an implantable pulse generator also placed in the patient.

Alternatively or additionally to any of the examples above, in another example, the implantable pulse generator may be in the left axilla, and the lead and electrode may be in the right ITV.

Alternatively or additionally to any of the examples above, in another example, the implantable pulse generator may be in the left axilla, and the lead and electrode may be in the left ITV.

Alternatively or additionally to any of the examples above, in another example, the implantable pulse generator may be placed in a subclavicular pectoral position on the patient's chest.

Alternatively or additionally to any of the examples above, in another example, the therapy may be a bradycardia pacing therapy.

Alternatively or additionally to any of the examples above, in another example, the therapy may be an anti-tachycardia pacing therapy.

Alternatively or additionally to any of the examples above, in another example, the therapy may be a cardiac resynchronization therapy.

Alternatively or additionally to any of the examples above, in another example, the second electrode may be also disposed in the mediastinum.

Alternatively or additionally to any of the examples above, in another example, both the first and second electrodes may be disposed on a single lead in the mediastinum adjacent to the right ITV.

Alternatively or additionally to any of the examples above, in another example, both the first and second electrodes may be disposed on a single lead in the mediastinum adjacent to the left ITV.

Alternatively or additionally to any of the examples above, in another example, the first electrode may be in the mediastinum adjacent to the right ITV, and the second electrode may be in the mediastinum adjacent to the left ITV.

Alternatively or additionally to any of the examples above, in another example, the second electrode may be disposed on an internal pulse generator also implanted in the patient.

Alternatively or additionally to any of the examples above, in another example, the implantable pulse generator may be in the left axilla, and the lead and electrode may be in the mediastinum adjacent to the right ITV.

Alternatively or additionally to any of the examples above, in another example, the implantable pulse generator may be in the left axilla, and the lead and electrode may be in the mediastinum adjacent to the left ITV.

Alternatively or additionally to any of the examples above, in another example, the implantable pulse generator may be placed in a subclavicular pectoral position on the patient's chest.

Alternatively or additionally to any of the examples above, in another example, the therapy may be a defibrillation therapy and both the first and second electrodes may be disposed on a single lead within the mediastinum adjacent to the same ITV.

Alternatively or additionally to any of the examples above, in another example, the therapy may be a defibrillation therapy and the second electrode may be disposed subcutaneously on a lead in the patient.

Alternatively or additionally to any of the examples above, in another example, the therapy may be a defibrillation therapy, wherein the first electrode may be electrically in common with a third electrode during the therapy delivery.

Alternatively or additionally to any of the examples above, in another example, the third electrode may be disposed in the mediastinum adjacent to the same ITV as the first electrode. Alternatively or additionally to any of the examples above, in another example, the third electrode may be disposed in the mediastinum adjacent to an ITV such that one of the first and third electrodes may be in the mediastinum adjacent to the right ITV, and the other of the first and third electrodes may be in the mediastinum adjacent to the left ITV.

Alternatively or additionally to any of the examples above, in another example, the first electrode may be a composite electrode including at least a first coil electrode electrically in common with a first ring electrode.

Alternatively or additionally to any of the examples above, in another example, the first electrode may be a composite electrode including at least first and second coil electrodes electrically in common with one another.

In another example, a method of implanting a lead for use in a cardiac stimulus system in a patient, the lead having at least one electrode thereon may comprise inserting a distal end of a lead into in a recess created in the mediastinum adjacent to the ITV, advancing the lead to a desired location relative to the heart of a patient, and securing the lead in place.

In another example, an implantation tool set may be configured for use in any of the examples herein.

In another example, an implantable cardiac stimulus device may comprise a lead and an implantable canister for coupling to the lead. The implantable canister may house operational circuitry configured to deliver output therapy in the form of at least one of bradycardia pacing, anti-tachycardia pacing, cardiac resynchronization therapy, or defibrillation, according to any of the examples herein.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A method of implanting a lead for use in a cardiac stimulus system in a patient, the lead having at least one electrode thereon; the method comprising:
entering the internal thoracic vein (ITV);
exiting the ITV to enter the mediastinum;
creating a recess in the mediastinum; and
inserting the lead into the recess created in the mediastinum to a desired location relative to the heart of a patient.

2. The method of claim 1 further comprising establishing access to a brachiocephalic vein of the patient and advancing a distal portion of the lead to the ITV from the brachiocephalic vein to enter the ITV.

3. The method of claim 1 wherein exiting the ITV to enter the mediastinum comprises puncturing a wall of the ITV.

4. The method of claim 3 wherein exiting the ITV to enter the mediastinum further comprises advancing a dilator with a sheath thereon through the puncture in the wall of the ITV into the mediastinum and removing the dilator.

5. The method of claim 4 wherein the step of creating a recess in the mediastinum comprises advancing the dilator into the mediastinum prior to removal of the dilator.

6. The method of claim 3 wherein the step of creating a recess in the mediastinum comprises advancing a balloon catheter having an expandable balloon through the puncture and into the mediastinum, and expanding the expandable balloon to compress a region of tissues in the mediastinum to create the recess.

7. The method of claim 6 wherein the step of advancing the balloon catheter is performed by first placing a guidewire through the puncture and into the mediastinum, and pushing the balloon catheter over the guidewire.

8. The method of claim 1 wherein entering the ITV comprises establishing access to the ITV through an intercostal space between two ribs.

9. The method of claim 8 wherein establishing access to the ITV through an intercostal space comprises:
inserting a needle into one of the ITV through the intercostal space; and
advancing a sheath into the intercostal space and into the ITV; and
wherein the step of inserting the lead comprises advancing the distal end of the lead through the sheath and into the ITV.

10. The method of claim 1 wherein entering the ITV comprises establishing access to the superior epigastric vein at a location inferior to the lower rib margin and advancing a lead superiorly into the ITV.

11. The method of claim 1 wherein entering the ITV comprises establishing access to the musculophrenic vein at about the lower rib margin and advancing a lead superiorly into the ITV.

12. The method of claim 1 further comprising anchoring the lead using an inflatable balloon, a lobe, a tine, a hook, or a stent.

13. The method of claim 1 wherein the lead is configured to have a curvature and the method further comprises anchoring the lead by allowing it to assume the curvature once implanted to a desired position.

14. A method as in claim 1 further comprising coupling the lead to an implantable pulse generator, and implanting the pulse generator at a subclavicular position.

15. A method as in claim 1 further comprising coupling the lead to an implantable pulse generator, and implanting the pulse generator at a left axillary position.

16. A method of treating a patient comprising delivering therapy between:
a first electrode disposed on a lead which is placed in a recess created in a mediastinum of a patient, a portion of the lead passing through an internal thoracic vein; and
at least a second electrode.

17. The method of claim 17 wherein the second electrode is disposed on an implantable pulse generator also placed in the patient.

18. The method of claim 17 wherein the second electrode is disposed on the same lead as the first electrode.

19. The method of claim 17 wherein the first electrode is disposed on a first lead, and the second electrode is disposed on a second lead different from the first lead.

20. A method of interacting between two implantable medical devices comprising:
issuing a communication using at least a first electrode disposed in the mediastinum, the first electrode being on a lead;
receiving the communication at a leadless implantable pacemaker disposed in the heart;
wherein the lead is disposed partly in the mediastinum and partly in an internal thoracic vein.
